(19) United States
(12) Patent Application Publication
(10) Pub. No.: US 2005/0002904 A1
(43) Pub. Date: Jan. 6, 2005

(54) Uses of vascular endothelial growth factor and type I collagen inducible protein (VCIP)

(76) Inventors: Kishore K. Wary, Houston, TX (US); Joseph O. Humtsoe, Houston, TX (US)

Correspondence Address: Dr. Benjamin Adler;ADLER & ASSOCIATES, 8011 Candle Lane, Houston, TX, 77071, US

(21) Appl. No.: 10/812,238

(22) Filed: Mar. 29, 2004

Related U.S. Application Data
(60) Provisional application No. 60/485,164, filed on Jul. 3, 2003.

(51) Int. Cl. A61K 48/00

(52) U.S. Cl. 424/93.2; 514/44

(57) ABSTRACT

Vascular endothelial growth factor and type I collagen inducible protein (VCIP), also known as phosphatidic acid phosphatase 2b (PAP2b), was identified in a functional assay of angiogenesis. Previously, VCIP was not known to function as an integrin ligand. The present invention discloses VCIP-derived peptides and proteins act as integrin ligands. Since VCIP-derived peptides or proteins are capable of inhibiting specific cell-cell interactions, such inhibitors of cell-cell interactions would be useful for developing novel therapeutic approaches to treat diseases where these interactions have clear pathological consequences. For example, VCIP/PAP2b can be a novel target for anti-angiogenic, anti-cancer and anti-metastatic therapy.

### CROSS-REFERENCE TO RELATED APPLICATION

This non-provisional patent application claims benefit of provisional patent application U.S. Ser. No. 60/458,164, filed Mar. 27, 2003, now abandoned.

### BACKGROUND OF THE INVENTION

1. Field of the Invention

The present invention relates generally to the field of cell-cell interaction. More specifically, the present invention discloses novel functions for vascular endothelial growth factor and type I collagen inducible protein (VCIP) in cell-cell interaction and intracellular signaling.

2. Description of the Related Art

Cell-cell and cell-matrix interactions play fundamental roles in embryonic development and in wound healing, and these interactions are known to be altered in many pathological processes. Endothelial cells, which line the walls of blood vessels, are able to promote both ‘homotypic’ and ‘heterotypic’ cell-cell interactions. Such interactions are critical for angiogenesis, which proceeds through several distinct coordinated steps. Initially, endothelial cells that are contact inhibited or considered to be in the G₀ phase of the cell cycle become activated in response to an increase in local concentrations of angiogenic factors. Activated endothelial cells then locally secrete proteases to dissolve basement membranes, thereby allowing endothelial cells to detach from the vascular wall. The detached endothelial cells then send out cytoplasmic projections, migrate, elongate extensively and form cell-cell interactions. Eventually, endothelial cells enter the cell cycle and can either differentiate into tube-like structures, depending upon the presence of specific survival factors and extracellular matrix (ECM) components, or undergo apoptosis, which can disrupt angiogenesis.

These in vivo processes can be partially duplicated in vitro by providing endothelial cells with appropriate extracellular matrix molecules and a gradient of angiogenic cytokines, such as basic fibroblast growth factor (bFGF) and vascular endothelial growth factor (VEGF). VEGF and bFGF can act on endothelial cells either individually or in a coordinated manner to transduce extracellular signals into distinct cellular transcriptional responses. The specific roles of VEGF and its receptors in angiogenesis have been well documented. While most of these angiogenic cytokines directly regulate normal angiogenesis, unrestrained production of these factors can potentially deregulate cell-cell interactions, cell-matrix interactions and gene expression. Such deregulation may contribute to various vascular abnormalities, including the growth of solid tumors, cardiovascular disease and diabetic retinopathy.

Activated endothelial cells detach from the endothelium and maintain cell-cell contact in order to survive; the absence of such cell-cell interactions can promote anoikis. Studies suggest that endothelial cells-mediated cell-cell interactions are also required for the recruitment of pericytes, as well as for the stabilization and maturation of blood vessels. Molecules that mediate cell-cell interactions include integrins and their ligands, VE-cadherin, PECAM-1 (CD31), junctional adhesion molecules (JAM), VCAM-1, selectins, claudins, Eph and Ephrins. These adhesion molecules are also involved in the assembly and formation of adherent and tight junctions, phenotypes that are closely associated with the formation of mature blood vessels and the segregation of arteries and veins.

Addition of angiogenic factors to quiescent endothelial cells cultured in three-dimensional type I collagen matrices induced capillary morphogenesis. Recently a set of 12 novel genes were identified from these endothelial cells undergoing capillary morphogenesis in three-dimensional collagen matrices. These 12 genes had not been previously reported to be associated with the processes of angiogenesis. One of these genes is designated VCIP for VEGF and type I collagen inducible protein (DDBJ/EMBL/GenBank accession No. AF480883), which is also known as phosphatidic acid phosphatase type 2b (PAP2b). Until now, no function other than lipid phosphatase activity has been described for VCIP.

The prior art is deficient in uses of vascular endothelial growth factor and type I collagen inducible protein in cell-cell interaction and intracellular signaling as well as pathophysiological states. The present invention fulfills this long-standing need and desire in the art.

### SUMMARY OF THE INVENTION

Vascular endothelial growth factor and type I collagen inducible protein (VCIP), also known as phosphatidic acid phosphatase 2b (PAP2b), was identified in a functional assay of angiogenesis. VCIP/PAP2b exhibits an Arg-Gly-Asp (RGD) cell adhesion sequence. Immunoprecipitation and fluorescence-activated cell sorting analyses demonstrated that VCIP-RGD is exposed to the outside of the cell surface. Retroviral transduction of VCIP induced cell aggregation/cell-cell interactions, modestly increased p120 catenin expression and promoted activation of the Fak, Akt and GSK3β protein kinases. Furthermore, expression of recombinant VCIP promoted adhesion, spreading and tyrosine phosphorylation of Fak, Shc, Cas and paxillin in endothelial cells. GST-VCIP-RGD, but not GST-VCIP-RGE, specifically interacted with a subset of integrins, and these interactions were effectively blocked by anti-αvβ3 and anti-α5β1 integrin antibodies, and by PAP2b/VCIP-derived peptides. Interestingly, PAP2b/VCIP is expressed in close proximity to vascular endothelial growth factor, von Willebrand factor and αvβ3 integrin in tumor vasculatures. These findings demonstrate an unexpected function of PAP2b/VCIP, and represent an important step towards understanding the molecular mechanisms by which PAP2b/VCIP-induced cell-cell interactions regulate specific intracellular signaling pathways.

The present invention also provides evidence that the cytoplasmic domain of VCIP interacts with p120catenin that alters β-catenin localization and LEF-1 transcriptional activation. In particular, retroviral-mediated over-expression of wild-type VCIP in primary endothelial cells impeded wound healing without affecting proliferative potential of these cells. Reciprocal co-immunoprecipitation and western immunoblot analyses showed that VCIP binds to p120catenin on endothelial cells, but not VE-cadherin or other Armadillo domain-containing proteins such as β-catenin or γ-catenin (plakoglobin). VCIP immunocomplex prepared from E-cadherin-deficient SW480 cell line contained p120catenin immunoreactivity, suggesting VCIP and p120catenin interaction may be E cadherin-independent. Moreover, a truncated VCIP without C-terminal cytoplasmic domain failed to coprecipitate p120catenin. Far-western analyses suggested that the cytoplasmic domain of VCIP interacts with p120catenin. Furthermore, it was demonstrated that elevated expression of VCIP in SW480 cells induced recruitment of p120catenin directly and promoted redistribution of β-catenin indirectly. These results are consistent with the observations that elevated expression of wild-type VCIP in SW480 cells caused increased cell-cell contact formation, decreased phosphorylation of β-catenin, and moderate inhibition of LEF-1-mediated transcription. Taken together, these results show that in endothelial and SW480 cells VCIP mediates cell-cell adhesion and modulates Wnt signaling pathway in an unprecedented manner.

The present invention further provides evidence that expression of VCIP potentiates tumor growth and metastasis in athymic nude mice by recruiting endothelial cells and regulating tumor angiogenesis. Human colorectal adenocarcinoma (SW480) cells stably expressing various human VCIP/PAP2b cDNA constructs were generated. These SW480 cells were xenografted subcutaneously into nude mice, and the role of VCIP in tumor growth, angiogenesis, and metastasis was monitored for a period of 30-45 days. Metastatic foci formation at distance sites were determined by the presence of human ALU DNA repetitive sequence in mouse tissue. Control SW480 parental cells were tumorigenic but did not grow beyond 2 mm in size. In contrast, SW480 cells expressing VCIP-RGD (wild-type) promoted aggressive tumor growth beyond 2 mm, accompanied by tumor neovasculature formation and induced metastases to brain, liver, and lung. Phosphatase inactive VCIP/PAP2b and delta-C-cyto mutants also promoted tumor growth and neovascularization, but did not support metastasis. This response was greatly diminished in SW480 cell expressing VCIP-RGE (mutant), as illustrated by the lack of neovascularization and metastasis. Furthermore, anti-VCIP/PAP2b-RGD antibody also significantly inhibited bFGF- and VEGF-induced experimental angiogenesis. Together, these data indicate that VCIP/PAP2b contains at least two distinct functional domains (i.e., phosphatase enzyme and RGD cell adhesion) and phosphatase function is not required for angiogenesis. Both domains were shown to act in synergy to potentiate tumor growth, angiogenesis and metastasis in an unprecedented manner. Thus, the present invention reveals VCIP/PAP2b as a novel potential target for anti-angiogenic, anti-cancer and anti-metastatic therapy.

In one embodiment of the present invention, there is provided a method of enhancing cell-cell interactions by over-expressing vascular endothelial growth factor and type I collagen inducible protein (VCIP) in a cell.

In another embodiment, there is provided a method of inhibiting cell-cell interactions by blocking the binding of integrin to cell surface VCIP.

The present invention also provides methods of treating a patient having a pathological condition resulted from integrin-mediated cell-cell interaction. The methods involve blocking the binding of integrin to cell surface VCIP by antibodies directed against a VCIP peptide comprising a RGD sequence or by a RGD-containing peptide derived from VCIP. Alternatively, the function of VCIP can be blocked by VCIP anti-sense oligonucleotides. In one embodiment, blocking integrin binding to VCIP can be used to inhibit angiogenesis and the formation of capillaries in a patient.

In yet another embodiment, the present invention provides peptides derived from VCIP, vectors encoding such peptides and antibodies directed against such peptides.

In still yet another embodiment, there are provided methods of using VCIP to enhance cell-cell adhesion junction formation in a patient or to enhance angiogenesis in a patient.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIGS. 1A-M show expression analysis and predicted amino acid sequence of VEGF and type I collagen inducible protein (VCIP). FIG. 1A: HUVECs were embedded into three-dimensional type I collagen gel in the presence of 20% adult human serum, 1× ITS and stimulated with VEGF¹⁶⁵ (100 ng/ml). Total RNA was prepared at the indicated time points. Northern blots were hybridized with a fragment of the VCIP (clone-33A) cDNA probe. The transcript size (3.4 kb) is indicated on the right. The numbers at the bottom of the gel represent the fold increase in VCIP mRNA levels, as compared with untreated cells. FIG. 1B: Ethidium bromide-stained agarose gel shows equivalent amounts of RNA used. FIG. 1C: HUVECs were cultured as above and left untreated, the last lane shows cells treated with VEGF for 12 h as a positive control. Total RNA was isolated at indicated time points and subjected to VCIP northern blot analysis. FIG. 1D: The blot shown in FIG. 1C was stripped and re-hybridized with a GAPDH probe to show that equal amounts of RNA were loaded across the lanes. FIG. 1E: Poly(A)⁺ RNA prepared from various human tissues was subjected to VCIP northern blot analysis by hybridization with the clone-33A cDNA probe. FIG. 1F: The blot shown in FIG. 1E was re-hybridized with a GAPDH probe, showing that equal amounts of RNA were loaded across the lanes. FIG. 1G: Detection of VCIP mRNA by RT-PCR (20 cycles): monolayer endothelial cells were treated for 30 min with: (1) untreated, (2) bFGF (20 ng/ml), (3) VEGF¹⁶⁵ (100 ng/ml), (4) PMA (20 ng/ml), (5) bFGF (20 ng/ml)+VEGF (100 ng/ml), (6) bFGF (20 ng/ml)+PMA (20 ng/ml), (7) VEGF (100 ng/ml)+PMA (20 ng/ml) and (8) bFGF (20 ng/ml)+VEGF (100 ng/ml)+PMA (20 ng/ml). FIGS. 1H-J: RT-PCR analysis of uPAR mRNA (FIG. 1H), β-actin mRNA (FIG. 11) and GAPDH mRNA levels (FIG. 1J) were carried out under the same conditions as described in FIG. 1G. All experiments were repeated at least three times, with similar results. FIG. 1K: Inserts from six recombinant_phage clones are shown, and the relative position of clone 33A is indicated. FIG. 1L: The complete predicted amino acid sequence of VCIP. The putative transmembrane segments are underlined. The N-linked glycosylation site is indicated by an asterisk. The lipid phosphatase catalytic domain is indicated by bold letters and underlined, and the RGD motif is boxed. FIG. 1M: The amino acid sequence of human VCIP (top) is aligned with other proteins containing lipid phosphatase-like catalytic motifs.

FIGS. 2A-C show expression of VCIP requires α2β1 integrin ligation and VEGF¹⁶⁵ treatment. Endothelial cells were embedded in 3D type I collagen for 12 hr and incubated in the presence or absence of VEGF, bFGF, and the indicated antibodies: 20.0 μg/ml of purified anti-α1β1 (TS2/7, ATCC), 20.0 μg/ml of purified anti-α2β1 integrin (MAB1998, Chemicon), 15.0 μg/ml anti-VEGF¹⁶⁵ (clone 26503.11, Sigma), and 15.0 μg/ml anti-bFGF (clone FB-8, Sigma) antibodies. Antibodies were dialyzed against sterile 1× TBS, pH 7.4 for 24 hours at 4° C. to remove traces of sodium azide and possible contaminants. Endothelial cells were pretreated with anti-α2β1 integrin and anti-VEGF¹⁶⁵ in Media M199+20% adult human serum prior to embedding into 3D type I collagen. Poly A+ mRNA was isolated and 2 μg/lane was subjected to northern blot analysis. Membrane was exposed for 120 hr at −70° C., so that the minimal expression of VCIP in unstimulated cells (first lane) could be detected (FIG. 2A). Northern blot signal intensities as quantified by PhosphorImager. By normalizing to the weakest hybridization signal (lane 1), the relative fold increases in mRNA expression were calculated (FIG. 2B). The histogram shows averaged results from two experiments. The membrane was stripped and reprobed with an uPAR probe as a control for the cytokines used (FIG. 2C).

FIGS. 3A-F show VCIP induction by growth factors and cytokines. Indicated monolayer cells were stimulated with various growth factors and cytokines for 6 h in media M199 containing 10% serum+1× ITS. The concentrations of cytokines used were optimized according to their ability to induce Erk2 phosphorylation in western blot analysis: VEGF¹⁶⁵ (100 ng/ml), EGF (20 ng/ml), bFGF (30 ng/ml), TNF-α (15 ng/ml), PMA (10 ng/ml) and IL-1β (25 ng/ml). Total RNA (20 μg per lane) was subjected to northern blot analysis by hybridization with the indicated probes. The uPAR northern blot was included as a control for the cytokines used. Ethidium bromide-stained gels show equal amounts of RNA used (FIGS. 3C and F). Data shown are representative of those obtained in two or three separate experiments.

FIGS. 4A-J show schematic diagrams of various recombinant cDNA constructs used in this study. FIGS. 4A-D: pEGFP-based constructs. FIGS. 4F-G: pLNCX2 retroviral constructs. FIGS. 4H-J: pGST-fusion protein constructs. The relative positions of RGD and RGE within the constructs are indicated. HA indicates hemaglutinin epitope tags. All constructs are shown in the 5′-3′ orientation. Arrows indicate the direction of transcription.

FIGS. 5A-C show VCIP is a cell surface antigen. HEK293 cells were transiently transfected with the indicated constructs and subjected to cell surface biotinylation. Cell extracts were then subjected to immunoblotting or immunoprecipitation. FIG. 5A: Anti-GFP immunoblotting. FIG. 5B: Immunoprecipitation with anti-GFP monoclonal antibodies followed by streptavidin-HRP immunoblotting. FIG. 5C: FACS analyses of HEK293 cells stably expressing the V (vector alone) or WT (wild-type) constructs. Both cells were stained with anti-VCIP-RGD mAb (30 μg/ml) followed by FITC-labeled anti-rabbit IgG antibodies. Data shown are representative of those obtained in at least three separate experiments.

FIGS. 6A-K show VCIP promotes cell-cell interactions. FIG. 6A: HEK293 cells were retrovirally transduced with either pLNCX2 (V cells, vector only), pLNCX2-VCIP-RGD (WT cells, wild-type) or pLNCX2-VCIP-RGE (MT cells, mutant) and were then propagated in medium containing G418 (500 μg/ml). Clarified cell extracts were immunoprecipitated with affinity purified anti-VCIP-c-cyto rabbit polyclonal antibodies and analyzed by anti-HA immunoblotting. FIGS. 6B-D: Formation of cell aggregates. Twenty thousand cells were deprived of growth factors and plated for 36 h in defined medium: V cells (FIG. 6B), WT cells (FIG. 6C), and MT cells (FIG. 6D). Photomicrographs revealed the presence of cell aggregates (indicated by arrowheads) after 12-24 h in WT cells, but not in V or MT cells. Bar, 25 mm. FIG. 6E: BrdU uptake was used to measure cell proliferation in V, WT and MT cells. Data are expressed as mean±SD from triplicate samples. All experiments were performed at least three times. FIGS. 6F-G: High-resolution photomicrographs of live WT cell aggregates after 3 days (FIG. 6F) or 5 days (FIG. 6G). Bar, 25 mm. FIG. 6H: The percentage of apoptotic nuclei was determined by scoring at least 300 cells from five randomly selected microscopic fields. Data are expressed as the mean±SD from triplicate samples. *, P<0.05. FIGS. 61-K: Representative photomicrographs of Hoechst-stained V cells (FIG. 6I), WT cells (FIG. 6J) and MT cells (FIG. 6K). Apoptotic nuclei are indicated by arrows.

FIGS. 7A-O show the effects of VCIP on adhesion of cadherin-deficient SW480 cells to HUVECs monolayer. SW480-C, SW480-WT, and SW480-MT cells were non-enzymatically detached from dishes, washed, passed through a cell strainer, counted, and labeled with a fluorescent dye (red) prior to experiments. HUVECs were seeded onto 12-well dishes in complete media and allowed to form a 100% confluent monolayer on the day of assay. Monolayer HUVECs were washed once each with PBS and HCMF, and then preincubated in HCMF supplemented with Ca²⁺/Mg²⁺ for 10 min at 37° C. SW480 cells (0.25×10⁶) were finely resuspendend in 500 μl of HCMF buffer supplemented with Ca²⁺/Mg²⁺, layered onto the monolayer, and allowed to attach at 37° C. for 1 hr. Unattached cells were removed by washing with PBS and adherent cells were fixed with 4% paraformaldehyde. The number of cells adhered to the monolayer were determined using a phase-contrast microscope. Experiments were performed at least three times in triplicates, with three independent clones. At least five random microscopic fields were selected for counting attached SW480 cells at 100× magnification The total number of SW480-WT cells attached to the monolayer during first three experiments was designated as 100% cell attachment. In each experiment, the relative percentage of cell adhesion was calculated and presented as mean±SD. Photomicrographs in the left panels show attachment of stable SW480 cells onto the monolyaer (tungsten light), which were shown out of focus to enhance the visualization of attaching SW480 cells. Photomicrographs in the right panel show fluorescently-labeled SW480 cells in the same microscopic fields. Bar 40 μm. Data are expressed as mean±S.D. (n≧7), *P<0.05. FIGS. 7A-B: HUVECs layered with SW480-C cells. FIGS. 7C-D: HUVECs layered with SW480-WT cells. FIGS. 7E-F: HUVECs layered with SW480-MT cells. FIGS. 7G-N: HUVECs layered with SW480-WT cells pre-incubated with 10 μM GRGDSP peptide (FIGS. 7G-H), 20 μM GRGDSP peptide (FIGS. 7I-J), 20 μg/ml affinity purified anti-VCIP-RGD antibody (FIGS. 7K-L), 40 μg/ml affinity purified anti-VCIP-RGD antibody (FIGS. 7M-N). All antibodies were dialyzed against sterile 1× TBS, pH 7.4 for 24 hours at 4° C. to remove traces of sodium azide and possible contaminants prior to use.

FIG. 7O: Histogram showing adhesion of SW480 cells in the presence or absence of indicated substances.

FIGS. 8A-G show VCIP-induced regulation of various intracellular signaling pathways. Cells were cultured exactly as described in FIG. 6. At the end of 36 h, V, WT and MT cells were solubilized, and extracts analyzed by immunoblotting with the indicated antibodies. Phospho-specific immunoblots were stripped and re-probed with the corresponding total antibodies to confirm that equal amounts of protein were loaded across the lanes as shown. All blots shown are representative of those obtained in at least three separate experiments. FIG. 8A: β1 integrin immunoblot. Arrowhead indicates immature form of the β1 integrin subunit. FIG. 8B: p120ctn immunoblot. HEK293 cells express two isoforms of p120ctn, isoform-1 (p120) and isoform-2 (p110); p110 is less abundant in HEK293 cells. FIG. 8C: Phospho-FAK and total FAK immunoblots. FIG. 8D: Phospho-Akt and total Akt immunoblots. FIG. 8E: Phospho-Jnk and total Jnk immunoblots. FIG. 8F: Phospho-Erk½ and total Erk½ immunoblots. FIG. 8G: Phospho-GSK3β and total GSK3β immunoblots.

FIGS. 9A-F show VCIP mediates heterophilic cell-cell interactions. Magnification, 200×. Bar, 40 mm. *, P<0.05. The images of cell aggregates appear out of focus. FIG. 9A: Representative photomicrograph of a mixture of WT and MT cells labeled with red (Dil) and green (DiO) fluorescent dyes at 0 h. FIG. 9B: Small and large cell-aggregates were visible after 6 h; green arrowheads indicate the most productive cell aggregation (yellow). FIG. 9C: Representative large cell aggregates are shown after 12 h. FIG. 9D: Cells co-incubated with the GRGDSP peptide for 12 h. FIG. 9E: Cells co-incubated with anti-VCIP-RGD antibody (25 μg/ml) after 12 h. All experiments were performed at least three times in triplicates. FIG. 9F: Histogram showing that the anti-VCIP-RGD (α-VCIP) antibody and GRGDSP (RGD-) peptides dose-dependently inhibited cell aggregation, whereas control antibodies (α-IgG) and peptides GRADSP (RAD-) did not.

FIGS. 10A-H shows recombinant expression of VCIP-RGD protein induces interactions with αvβ3 and α5β1 integrins. FIG. 10A: Affinity purified GST-fusion proteins were resolved on SDS±PAGE and stained with Coomassie Blue. FIG. 10B: Integrins were immobilized onto 96-well plates, and various GST-fusion proteins (ligands) were allowed to bind. Bound ligands were incubated with anti-GST antibodies, appropriate substrate added and the color intensity measured. OD values <0.05 were considered to be background binding. Data are expressed as mean±SD (n=4). FIG. 10C: Dose-dependent binding of αvβ3 integrin with recombinant GST-VCIP-RGD. FIGS. 10D-F: Representative photomicrographs of adhesion and spreading of endothelial cells replated onto dishes coated with either fibronectin (FIG. 9D), vitronectin (FIG. 9E) or GST-VCIP-RGD (FIG. 9F). Cells were allowed to attach for 45 min, washed with PBS and stained with H&E. Magnification, 100×. Bar, 25 μm. FIG. 10G: Adhesion blocking assays. Endothelial cells were replated onto dishes coated with increasing concentrations of affinity purified fusion proteins (1, 5 and 10 nM). Attached cells were stained, washed and absorbances of eluted dyes measured. To determine the effects of anti-integrin antibodies, dishes were coated with 10 nM GST-VCIP-RGD. Endothelial cells were pre-incubated at 4° C. with increasing concentrations of anti-α5β1 (P1D6), anti-αvβ3 (LM609), anti-α2β1 (MAB 1998) and anti-α3β1 (P1B5) monoclonal antibodies (1, 5 and 10 μg/ml) for 30 min, then washed and replated onto coated dishes. Attached cells were stained, washed and absorbances of eluted dyes determined. Three independent experiments were performed with several replicate samples in each experiment. Data are expressed as mean±S.D.; diamond, P<0.01; asterisk, P<0.001. FIG. 10H: Clarified extracts prepared from [³⁵S]Cys/Met-labeled HdMVEC were pre-adsorbed and incubated with GST-VCIP-RGD in the absence (−) or presence (+) of GRGDSP peptide (lanes 1 and 2). The presence of αvβ3 and α5β1 integrins (lanes 5 and 6) in GST-VCIP pull-down complex (lane 1) was confirmed by incubating aliquots of dissociated complex with the indicated antibodies. Anti-mouse IgG was included as negative control (lane 3).

FIGS. 11A-G shows tyrosine phosphorylation of Fak, Cas, Shc, Paxillin and Erk2. Dishes were coated with either fibronectin (Fn, 10 μg/ml), vitronectin (Vn, 10 μg/ml) or affinity purified GST-VCIP-RGD or -RGE fusion proteins (10 and 20 nM). Endothelial cells were detached, washed and maintained in suspension (Sus) for 30 min, then allowed to re-attach for 30 or 60 min. Adherent cells were solubilized in RIPA buffer. Blots shown are representative of those obtained in at least three separate experiments. FIGS. 11A-E: Equal amounts of protein (˜1.0 mg) from clarified extracts were subjected to immunoprecipitation (IP) followed by immunoblotting using the indicated antibodies. FIG. 11F: Equal amounts of protein from total lysates (35 μg/lane) were subjected to immunoblotting with the anti-phospho-Erk½ antibody. FIG. 11G: The same membrane was stripped and re-probed with an anti-Erk½ antibody, showing that equivalent amounts of proteins used across the lanes.

FIGS. 12A-I shows co-expression of VCIP with VEGF, and αvβ3 integrin in the tumor vasculature. Paraffin-embedded skin melanoma (FIGS. 12A-F) and angioma (FIGS. 12 G-I) tumor tissue sections (4 μm) were subjected to indirect double immunostaining. The sections were sequentially incubated with affinity purified anti-VCIP-RGD (30 μg/ml) (FIGS. 12A, D and G), anti-VEGF (FIG. 12B), anti-vWF (FIG. 12E) or anti-αvβ3 integrin (FIG. 12H) monoclonal antibodies. Sections were washed and incubated with goat anti-rabbit IgG conjugated to Texas Red (red) and goat anti-mouse IgG conjugated to FITC (green). Images were taken below the saturation level with a Zeiss Axiovert-125 epifluorescent microscope equipped with a camera. FIGS. 12C, F and I represent merged images of FIGS. 12A-B, FIGS. 12D-E, and FIGS. 12G-H, respectively. Co-expression and co-incidences are indicated in yellow. Magnification, 100×. L, lumen. Bar, 25 μm.

FIGS. 13A-D show quiescent endothelium lacks VCIP expression. Normal skin tissue section (4 μm) was processed as described above. Bright light photomicrograph of a skin section shows the architecture of normal tissue (FIGS. 13A). Sections were sequentially incubated with anti-VCIP-RGD (30 μg/ml) (FIG. 13B) and anti-CD31/PECAM-1 antibodies (20 μg/ml) (FIGS. 13C). Sections were then washed and incubated with goat anti-rabbit IgG conjugated to Texas red (red) and goat anti-mouse IgG conjugated to FITC (green). Images were taken below saturation level with a Zeiss Axiovert-125 epifluorescent microscope equipped with a camera. FIG. 13D represents the merged images of B and C. Images shown are representative of those obtained in at least three separate experiments. Magnification 100×. L, lumen. Bar, 25 μm.

FIGS. 14A-H show schematic diagrams of various pLNCX2 retroviral constructs used in FIGS. 14-19. The relative positions of RGD, RGE, and RAD within the constructs are indicated. Arrows indicate the direction of transcription (5′-3′). Asterisk indicates the location of lipid phosphatase-dead motif. FIG. 14P shows expression analysis of PAP2b/VCIP constructs in HUVECs. Whole cell extracts were analyzed by immunoprecipitation and western immunoblotting with anti-HA monoclonal antibodies (mAb). Arrowhead indicates IgG-heavy chain. Epitope tags: HA, hemaglutinin; FLAG, Flag-C2.

FIGS. 15A-K show retroviral infection of HUVECs with VCIP-RGD impedes their ability to migrate and close an artificial wound. HUVECs (70% confluent) in complete media were transiently infected with control (V), wild-type-PAP2b-RGD (WT), or mutant-PAP2b-RGD (MT) retroviral particles. After 24 hours post-infection, cells were washed once with PBS. Aliquots of cells were solubilized, expression of PAP2b/VCIP was analyzed by immunoprecipitation and western immunoblotting with indicated antibodies (FIG. 15A). The cells were analyzed for their ability to proliferate (FIG. 15B). Artificial wounds were introduced with a micropipete tip, washed with PBS to remove detached cells, and incubated in defined media (M199+1×ITS+100 ng/ml VEGF). Wound closure was monitored for 0 (FIGS. 15C, F, I), 5 (FIGS. 15D, G, J), and 10 (FIGS. 15E, H, K) hours. Experiments were performed in triplicates and repeated at least three times. *P<0.05. Magnification, 100×. Bar, 25 μm.

FIGS. 16A-K show PAP2b interacts with p120 catenin. HUVECs were infected with pLNCX2-PAP2b-RGD (WT) retroviral particles and allowed to recover for 36 hours in complete media before stimulated with VEGF¹⁶⁵ for 6 hours. Cells were solubilized in modified RIPA, clarified lysates were immunoprecipitated with indicated antibodies and analyzed by western immunoblotting. FIG. 16A: Immunoblotting with anti-p120 catenin (1.0 μg/ml). FIG. 16B: Immunoblotting with anti-HA (0.5 μg/ml). FIG. 16C: Immunoblotting with anti-VE-cadherin (lane 1), anti-β-catenin (lane 2), anti-γ-catenin (lane 3). FIGS. 16D-K: SW480 stably expressing wild-type PAP2b/VCIP (construct B) was solubilized in modified RIPA buffer and immunodepleted with anti-pan-cadherin monoclonal antibodies for 2 hours at 4° C. Lysate was recovered and absorbed with sepharose protein-G beads for 1 hour at 4° C. to eliminate left-over (anti-pan-cadherin) IgG. Immunodepleted samples were immunoprecipitated with indicated antibodies and analyzed as shown: immunoblotting with anti-p120 catenin (FIG. 16D), anti-β-catenin (FIG. 16E), anti-γ-catenin (1.5 μg/ml) monoclonal (FIG. 16F), anti-PAP2b-cyto (0.5 μg/ml) polyclonal antibodies (FIGS. 16G, H, I). Arrows indicate major isoforms of p120 catenin polypeptides. Arrowheads indicate PAP2b polypeptides (FIGS. 16G, H, I). All blots shown are representative of those obtained in at least three separate experiments.

FIGS. 17A-H show interaction of PAP2b/VCIP with p120 catenin in endothelial cells. FIGS. 17A-B: Clarified cell lysates were prepared from endothelial cells infected with indicated PAP2b retroviral constructs A-E as shown in FIG. 14 and subjected to immunoprecipitation with: anti-HA (lanes 1, 2, 4, 5 and 6) or anti-p120 catenin (lane 3) and analyzed by immunoblotting with the indicated antibodies. FIGS. 17C-H: Far western analysis. Endothelial cells were solubilized in RIPA buffer, and immunoprecipitated using indicated antibodies (5 μg/1.5 mg protein). The membrane shown in FIG. 17C was incubated with 2 μg/ml of Gst-PAP2b-cyto fusion protein for 1 hour, washed with 1×TBS+tween (0.1%) and analyzed by immunoblotting with anti-Gst monoclonal antibodies (2 μg/ml). Individual lanes from the blot was excised and probed with: anti-VE-cadherin (FIG. 17D), anti-p120ctn (FIG. 17E), anti-β-catenin (FIG. 17F), anti-γ-catenin (FIG. 17G), and β1-integrin (FIG. 17H) monoclonal antibodies. Arrows indicate major isoforms of p120ctn. White asterisks indicate unprocessed PAP2b polypeptide, while N-glycosylated PAP2b proteins appear as smear. All blots shown are representative of those obtained in at least three separate experiments.

FIGS. 18A-K show regulation of p120ctn and β-catenin by PAP2b/VCIP. Data shown are representative of those obtained in at least three separate experiments. Magnification, 100× (SW480), 200× (HUVECs). FIGS. 18A-B: SW480 E-cadherin-deficient cells do not express PAP2b as illustrated by negative staining (FIG. 18A) while p120-_-catenin (p120ctn) is diffusedly distributed in the cytoplasm (FIG. 18B) and β-catenin is mostly nuclear (FIG. 18C). FIGS. 18D-I: SW480 cells stably expressing WTPAP2b-RGD induce formation of cell-cell junction like structures. Cells were sequentially incubated with indicated antibodies, followed by secondary donkey IgGs conjugated either to FITC (green) or Texas-Red (red) (FIGS. 18D, E and G, H). FIGS. 18F and I are merged images of FIGS. 18D, E and G, H respectively. FIGS. 18J-K: HUVECs were infected with control construct pLNCX2 (FIG. 18J) or pLNCX2-PAP2b-RGD (FIG. 18K). Recruitment of p120ctn was analyzed by immunostaining with anti-p120ctn monoclonal antibody.

FIG. 19A shows LEF-1 transcription assay. SW480 cells stably expressing the indicated retroviral constructs A-F (FIG. 14) were transiently transfected with TOPFLASH (5 μg/10⁶ cells) together with tracer amount of β-galactosidase plasmids (1 μg/10⁶ cells) for 3 hours with SuperFect™ (Qiagen) and allowed to recover for 36 hours in complete media containing 5% fetal calf serum. The amount of proteins and β-galactosidase activity were determined. Protein concentrations were adjusted for equivalent β-gal activities. LEF-1 luciferase activities were measured by Luciferase Assay System (Promega). Experiments were performed in duplicate, using triplicate wells in each case and data are expressed as mean±SD. *p<0.05; **<0.01. FIGS. 19B-E show immunoblotting with anti-phosphospecific antibodies against the Ser-33 and Thr-37/47 amino acid residues. Lystates from SW480 cells stably expressing the indicated retroviral constructs A-F (FIG. 14) were probed with anti-phospo-β-catenin (FIG. 19B), anti-3-catenin (FIG. 19C), anti-phospho-GSK-3,1 (FIG. 19D) and anti-GRB-2 (FIG. 19E) antibodies. Over-expression of wild-type PAP2b in SW480 cells significantly reduced phosphorylation of 1-catenin (FIG. 19B, lane 2). SW480 cells expressing construct-C, D, F and E (lane 3, 4, 5 and 6) showed moderate increase in the phosphorylation state of 1-catenin, but not significantly higher than basal level (lane 1). In addition, over-exression of wild-type PAP2b (construct B) induced increased phosphorylation of GSK30 (Ser9) kinase protein at least by -2.5 fold (FIG. 19D, lane 2). In contrast, there was no significant alteration in the phosphorylation state of GSK3β in lanes 1, 3, 4, 5 and 6 (FIG. 19D). Equal loading of proteins were determined by immunoblotting with anti-GRB-2 antibody (FIG. 19E).

FIGS. 20A-O show the effects of PAP2b/VCIP expression on tumor growth in athymic nude mice. Female nude mice (3-4 weeks old) were injected subcutaneously with SW480 cells (˜2×10⁴ ) expressing indicated PAP2b constructs: vector alone control, PAP2b-RGD wild-type, PAP2b-RGE mutant, PAP2b-C-cyto and PAP2b-PD phosphatase dead mutant. After 30 days, the visible primary tumor outgrowth was photographed. FIG. 20F shows no visible growth of SW480 control cells beyond 2 mm in diameter. FIGS. 20G-J show primary tumors beyond 2-3 mm in size. FIGS. 20K-O show close-up images of experimental tumor growth in the athymic nude mice.

FIGS. 21A-E show detection of human ALU sequences in tissues from athymic nude mice injected subcutaneously with SW480 cells expressing indicated PAP2b constructs: vector alone control (FIG. 21A), PAP2b-RGD wild-type (FIG. 21B), PAP2b-RGE mutant (FIG. 21C), PAP2b-C-cyto (FIG. 21D) and PAP2b-PD phosphatase dead mutant (FIG. 21E). Genomic DNA (10 ng) from different tissues of the tumor-bearing mice were analysed for the presence of human ALU sequence by PCR. An internal control of mouse-glyceraldehyde-3-phosphate dehydrogenase (m-GAPDH) was included. As an external positive control for human ALU, genomic DNA extracted from SW480 was included.

FIG. 22 shows anti-VCIP antibody blocks angiogenesis in vitro. Antibodies were dialyzed in sterile dialysis buffer (25 mM Tris, 175 mM sodium chloride, 2 mM potassium chloride pH 7.4) overnight to remove traces of azide and impurities. Integrity of antibodies was determined by SDS-PAGE. Activation and capillary morphogenesis of endothelial cells were performed in a 3D type I collagen matrix as described previously (Humtsoe et al., 2003). To determine the effects of specific antibodies on pre-formed capillaries, the method of Bayless et al. was used. Briefly, at 24 hours, mAbs were added to the culture medium at a concentration of 20 μg/ml. Fresh mAbs were added every 12 hours for a total of 60 hours. To quantify the degree of capillary formation, 3D matrices were fixed at 24, 36, 48, 60, and 72 hours by aspirating the medium, washing with PBS, and then fixing with 4% glutaraldehyde in PBS, pH 7.4, overnight at 4° C. Matrices were then washed with distilled water and embedded in paraffin according to the manufacturer's instructions (Richard Allen Scientific). Serial sections (4 μm) were prepared, dehydrated, stained with acidified eosin, and destained with distilled water. Capillaries were counted and photographed using a Zeiss Axiovert 25 C microscope at 100× magnification. Each capillary tubule was surrounded by least 2 to 5 endothelial cells. Capillary formation was defined as the induction of a minimum of 3 separate capillary events within a single field. At least 5 random fields were counted for each sample. Experiments were performed in duplicate, using triplicate wells in each case, and results were expressed as mean±SEM. *p<0.05; p<0.01.

FIGS. 23A-J are representative photomicrographs showing effects of specific mAbs on pre-formed capillaries. Endothelial cells were cultured in 3D collagen matrices in the presence of VEGF¹⁶⁵. Cultures were treated with mAbs at 24 hours and at various time points indicated, the cultures were fixed, sections prepared, stained with eosin, and the number of capillaries counted as described above. The upper panels (FIGS. 23A-E) were treated with anti-MHC class II mAbs, whereas the lower panels (FIGS. 23H-J) were treated with anti-αᵥβ₃ integrin mAbs. Bar, 50 μM.

### DETAILED DESCRIPTION OF THE INVENTION

VCIP/PAP2b mRNA was identified as a 3.4 kb transcript, not as a 1.6 kb transcript as described previously (Kai et al., 1997). No 1.6 kb PAP2b transcript was detected in any of the northern blot analyses described below. The cell membrane fraction prepared from 293T cells over-expressing PAP2b showed phosphatase activity against phosphatidic acid that was independent of Mg²⁺, insensitive to N-ethylmaleimide exposure, and blocked by propranolol and sphingosine. Data disclosed below show that VEGF, bFGF and PMA are able to induce expression of VCIP in three dimensional as well as monolayer cells. Cell surface biotinylation and FACS data indicated that VCIP is located on the cell surface.

Retroviral-mediated elevated expression of wild-type VCIP in primary endothelial cells impeded cell migration and wound healing without altering proliferative potential of these cells. This observation suggested that VCIP might form a molecular complex on endothelial cells. A recent study showed cell-cell and basolateral sorting of VCIP (hLLP3) protein on polarized MDCK cells, while PAP2a (hLPP1) protein sorted on the apical surface. In these cells, the ecto-enzymatic activity of PAP2a remained intact, while PAP2b activity was markedly reduced. These studies also found that PAP2b contains a dityrosine (Y109/Y110) basolateral targeting motif that was first characterized in LDL receptor. The apical sorting of PAP2a is driven by the FDKTRL amino acid sequence, a similar motif that also occurs in cysteic fibrosis protein. Thus, it is possible that basolateral and cell-cell localization of VCIP serves as mechanisms to promote integrin ligation at the cell-cell junction. Many cell surface proteins have been localized in cell-cell junctions, and the existence of PAP2b-mediated cell-cell junctions in vivo can be examined by electron microscopy analyses. In addition, investigation into the effects of mediators of inflammation =as well as ischemia, SIP, CIP, LPA, thrombin that interfere with inter-endothelial cell junction functioning should provide insights into the role of PAP2b in cell-cell contact formation and disassembly including signaling through the EDG receptor pathways and blood vessel maturation.

Results disclosed below indicate that VCIP mediates cell-cell interactions and promotes phosphorylation of GSK30 and cAKT protein kinases. Furthermore, the C-terminal of VCIP directly associates with p120catenin, which is likely to affect Wnt signaling pathway. The removal of the C-terminal tail of VCIP abolishes interaction with p120catenin. Increased expression of VCIP stabilizes β-catenin in the cytoplasm and inhibits transcriptional activities through LEF-1. Removal of C-terminal cytoplasmic segment of VCIP augments LEF-1 transcriptional activities. The role of VCIP in angiogenesis can be further elucidated by structure-functional studies.

VCIP exhibits an RGD sequence, and it promotes heterophilic cell-cell interactions and signaling. Until now, no function other than lipid phosphatase activity has been described for VCIP. The present invention clearly shows that recombinant VCIP-RGD molecule can act as an integrin ligand in vitro. The present invention also demonstrates that the intact RGD motif of VCIP is a potent ligand for a subset of integrins. VCIP appears to be preferentially expressed in inflamed/angiogenic tissues. In addition to its known lipid phosphatase activity, it is proposed that VCIP promotes ‘heterophilic interactions’, in that it can mediate both “homotypic” (like) and “heterotypic” (unlike) cell adhesions. For example, VCIP-RGD could bind monocytes, and thereby enhance the adherence of neutrophils to endothelial cell monolayers. β1 and β2 integrins are known to mediate adherence of monocytes to endothelial and epithelial cells, an early event in the acute inflammatory response. It is also possible that activated endothelial cells could recruit carcinoma cells that express VCIP. Alternatively, carcinoma cells such as A43 1-like cells may utilize VCIP-RGD to recruit activated endothelial cells. Platelet integrin αIIbβ3 may also interact with VCIP-RGD and contribute to platelet adhesion and aggregation. Lateral cell-cell interactions may provide a mechanism to impede or stop further migration of cells, thereby sequestering a subset of integrins from the basolateral surface of the cells towards cell-cell junctions. While interactions of endothelial cells with mesenchymal or smooth muscle cells may serve as a mechanism to promote recruitment of mural cells or pericytes, this may also promote maturation of blood vessels.

In summary, the present invention identifies novel functions of PAP2b/VCIP. Since synthetic peptide and fusion proteins modeled after the second extracellular loop of VCIP bind selectively to αvβ3 and α5β1 integrins, VCIP-derived peptides or proteins should inhibit specific cell-cell interactions. Such inhibitors of cell-cell interactions could be useful for developing novel therapeutic approaches to treat diseases where these interactions have clear pathological consequences, such as inflammation, thrombosis, atherosclerosis, restenosis and tumor-induced angiogenesis. Experiments can be designed to identify other molecules that may directly or indirectly function with VCIP, and examine how these factors may influence cell-cell interactions.

The present invention provides methods of enhancing or inhibiting cell-cell interaction by modulating the expression and function of VCIP (SEQ ID NO: 13). In general, such cell-cell interaction contributes to a biological process such as normal cell cycle progression, unwanted cell cycle progression, vascular malformation, expansion of atherosclerotic lesions, invasion of tumor cells, inflammation, cell motility, or angiogenesis. Preferably, the cell-cell interaction is mediated by integrins. In one embodiment, VCIP expression can be enhanced by over-expressing VCIP in a cell, resulting in enhanced cell-cell interaction. A gene encoding VCIP can be delivered to a cell by methods generally known in the art. For example, gene delivery can be accomplished by a viral vector such as adenoviral vector or by a non-viral gene delivery system such as high pressure gene delivery system (“Genegun”) or liposome. Over-expression of VCIP may promote cell-cell adhesion junction formation in patients with compromised blood-brain barrier functions. This therapeutic approach may stop edema and hemorrhage following traumatic brain injury such as gun shot wound. Over-expression of VCIP may also enhance angiogenesis in patients who need growth of new blood vessels for treating various ischemic diseases.

In another embodiment, the function of VCIP and cell-cell interactions can be inhibited by blocking the binding of integrin to cell surface VCIP. Binding of VCIP to its ligand can be blocked by antibodies directed against a VCIP peptide comprising a RGD sequence or by a RGD-containing peptide derived from VCIP. The function of VCIP can also be blocked by anti-sense VCIP oligonucleotides. These methods of blocking and inhibiting the binding of VCIP are useful in treating an individual having a pathological condition resulted from undesirable integrin-mediated cell-cell interaction. In general, such pathological condition includes, but is not limited to, stroke, thrombosis, tumor growth, metastasis, arthritis, cardiac infarction, psoriasis, diabetic retinopathy, inflammation, and angiogenesis.

In yet another embodiment, the present invention provides a fragment of VCIP that contains a RGD sequence. Such peptide is useful in inhibiting the binding of VCIP to its ligand. Representative examples of such peptides include, but is not limited to, SEQ ID NO: 20 and 32. The present invention further provides vectors encoding such peptides as well as antibodies directed against such peptides. These antibodies can be incorporated into a kit useful for detecting VCIP in an individual having a disease such as pathological angiogenesis, inflammation, arthritis, psoriasis, atherosclerosis, or metastatic disease. The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Cells and Reagents

Human umbilical vein endothelial cells (HUVECs), human dermal microvascular endothelial cells (HdMVECs), carotid artery smooth muscle cells (CASMCs) and aortic smooth muscle cells (AoSMC) were obtained from Clonetics. ECM molecules, endotoxin-free fetal bovine serum, antibiotics, heparin, 100× ITS (insulin, transferrin and selenium), M199 media, anti-α5β1 (P1D6) and anti-α3β1 (P1B5) antibodies and Superscript II reverse transcriptase enzyme were obtained from Invitrogen. Basic Fibroblast growth factor (bFGF) and human recombinant vascular endothelial growth factor (hrVEGF¹⁶⁵) were purchased from R&D systems. Bovine skin-derived type I collagen (3.0 mg/ml) solution was purchased from Cohesion Inc. Multiple tissue northern blot, cDNA amplification kit and human placental cDNA library in _Triple-Ex vector were purchased from Clontech Laboratories, Inc. Anti-phosphospecific antibodies were purchased from New England Biolabs. Hybridomas producing the anti-human α1β1 integrin antibody (clone TS2/7) were obtained from ATCC. Anti-α2β1 (MAB 1998), anti-avb3 (LM609) and VE-cadherin (MAB1989) antibodies were procured from Chemicon. Mouse anti-p120catenin (clone 15D2) monoclonal antibody was obtained from Zymed Laboratories, Inc. Synthetic peptides LSPVDIIDRNNHHNM (SEQ ID NO:1) and EGYIQNYRCRGDDSKVQEAR (SEQ ID NO:2) were used to raise anti-VCIP-cyto-C16 and anti-VCIP-RGD antibodies, respectively (Alpha Diagnostic International). These antibodies were affinity purified prior to use.

### EXAMPLE 2

Monolayer and Three-Dimensional Cell Culture

Monolayer cell cultures were carried out as described previously. Three-dimensional matrix gel was prepared by gently mixing a cold solution of bovine skin-derived type I collagen solution (2.1 mg/ml) with media M199, 1× ITS, hrVEGF¹⁶⁵ (100 μg/ml) and glutamine (2.4 mM). The pH was adjusted to 7.5 with 0.1 N sodium hydroxide and sterile water was used to adjust the final volume. Proliferating endothelial cells in the third or fourth passage were cultured in complete media and gently resuspended in complete M199 media at a concentration of 4×10⁵ cells/ml. Twenty four-well tissue culture dishes were filled with 300 μl of cold 3D gel solution, and placed at 37° C. in a CO₂ incubator for 30-45 min to polymerize and solidify. Resuspended cells (2×10⁵ cells in 500 μl) were seeded onto 3D gel and the dishes were returned to the CO₂ incubator at 37° C. to allow the cells to attach for 2-3 h. At the end of this period, unattached cells were removed, and a second layer of 3D gel was poured that included M199 media supplemented with 20% adult human serum-AB and 2.4 mM L-glutamine, in the presence or absence of 100 ng/ml human recombinant VEGF¹⁶⁵. Thus, endothelial cells were grown embedded between two layers of type I collagen gel.

To induce capillary morphogenesis of endothelial cells, 3D gels were filled with 500 μl of tubulogenic media, including M199 media, 1× ITS, 20% adult human serum-AB and hrVEGF¹⁶⁵ (100 ng/ml). The term ‘tubulogenic media’ is used to describe the media that induces formation of ‘capillary (or tubule) morphogenesis’ of endothelial cells grown in 3D gels.

### EXAMPLE 3

cDNA Library Screening, Northern Blot Analysis, PCR and RT-PCR

A _TripleEx phage cDNA library prepared from human placenta (Clontech) was screened as described previously (Wary et al., 1993). Plasmids were extracted, purified by Qiagen affinity column and then digested with EcoRI and XbaI to confirm the presence of the insert. Six overlapping clones were subjected to DNA sequencing. All northern blot analyses were performed as described previously (Wary et al., 1993). In brief, 20 μg of total RNA or 2 μg poly(A)+ mRNA from control cells (i.e. endothelial cells embedded in three-dimensional type I collagen in the presence of 20% human adult serum-AB±100 ng/ml hrVEGF¹⁶⁵ supplied every 6 h) were fractionated on an agarose gel containing formaldehyde. To analyze various mRNA levels by RT-PCR, the following primers were used: VCIP-forward 5′-GGAGGATCCCTCGCGCCGCAGCCAGCGCCATGC-3′ (SEQ ID NO:3) and -reverse 5′-GTGGCACCTACATCATGTTGTGGTG-3′ (SEQ ID NO:4); human uPAR-forward 5′-CTTCCTGAAATGCGTCAACACC-3′ (SEQ ID NO:5) and -reverse 5′-TCATAGCTGGGAAAACTGAGGC-3′ (accession No. X51675) (SEQ ID NO:6); β-actin-forward 5′-GGCTGTGCTATCCCTGTACGCC-3′ (SEQ ID NO:7) and -reverse 5′-GGGCAGTGATCTCCTTCTGCAT-3′ (accession No. X00351) (SEQ ID NO:8); GAPDH-forward 5′-GGTCTCCTCTGACTTCAACAGCG-3′ (SEQ ID NO:9) and -reverse 5′-GGTACTTTATTGATGGTACATGAC-3′ (accession No. M33197) (SEQ ID NO:10). PCR, RT±PCR and probe preparation were carried out as described previously (Wary et al., 1993).

### EXAMPLE 4

Biochemical Methods

For western blot analysis, cells were washed with cold PBS, and solubilized in modified RIPA buffer (50 mM HEPES pH 7.5, 1.0% Triton X-100, 0.1% SDS, 0.25% deoxycholate, 150 mM sodium chloride, 1 mM EDTA, 25 mM sodium fluoride, 1 mM sodium pyrophosphate, 2 mM sodium orthovanadate and appropriate concentrations of various protease inhibitors). For cell surface biotinylation, HEK293 cells (5×10⁶) were transfected with either pEGFP-C3, pEGFP-N3, pEGFP-C3-VCIP or pEGFP-N3-VCIP using Superfect-Liposome™ (Qiagen). Biotinylation of cell surface proteins was carried out according to published procedures (Gottardi et al., 1995). Immunoprecipitation, immunoblotting and immunodetection protocols were all performed as described previously (Mainiero et al., 1995, 1997; Wary et al., 1996, 1998, 1999a,b).

### EXAMPLE 5

[³⁵S]Cys/Met Labeling of HdMVEC And Affinity Chromatography

Human dermal microvascular endothelial cells (HdMVECs) (3×10⁷) were deprived of growth factors in Cys/Met-free DMEM for 8 h. The cells were then incubated with 3 mCi of [³⁵S]Cys/Met (specific activity 1170.0 Ci/mmol) for 3 h at 37° C. in Cys/Met-free media in the presence of 1× ITS. After 3 h, the cells were rinsed twice with complete media and allowed to recover in complete media for 1 h at 37° C. The cells were then washed and solubilized in 4 ml of complete cell extraction buffer (CCEB: 50 mM HEPES pH 7.4, 150 mM sodium chloride, 1% Triton X-100, 0.1% β-octylglucoside, 1 mM MgCl₂, 2 mM CaCl₂, with freshly added 2 mM PMSF, 10 μg/ml aprotinin, 5 μg/ml leupeptin and 10 μg/ml pepstatin-A as protease inhibitors). Cell extracts were clarified, pre-adsorbed once with 1.5 ml of packed Sepharose beads coupled to GST-fusion proteins (2 mg/ml) and once with 1.0 ml (packed) anti-mouse IgG agarose for 2 h each at 4° C.

Pre-adsorbed lysates were divided into two tubes, and 7 μg of GST-VCIP-RGD fusion protein was added to each sample. One of the tubes included 25 μM of the synthetic soluble peptide GRGDSP (SEQ ID NO:11) which is known to disrupt α5β1 integrin-fibronectin interaction. GST-pull down was carried out at 4° C. for 8 h, complexes washed once with CCEB, three times with GST-fusion protein wash buffer (50 mM HEPES pH 7.4, 150 mM sodium fluoride, 5% glycerol, 0.5% NP-40, 1 mM CaCl₂ and 1 mM MgCl₂) and one final wash with 1× TBS pH 7.4.

The contents of the other tube were resuspended in 0.5 ml of dissociation buffer (10 mM Tris pH 7.4, 0.75% SDS, 1% Triton X-100 and 250 mM NaCl), boiled for 10 min, centrifuged immediately and the beads were discarded. The supernatant was diluted with 4 ml of dilution buffer: 10 mM Tris pH 7.4, 100 mM NaCl, 1.0% Triton X-100, 2 mM CaCl₂ and 2 mM MgCl₂. This was equally divided into tubes containing 5 μg of either anti-mouse IgG, anti-αvβ3 (LM609) or anti-α5β1 (P1D6) integrin monoclonal antibodies. Immunoprecipitates were washed three times with cold CCEB and once with cold 1× TBS pH 7.4. Samples were boiled in non-reducing sample buffer and resolved by SDS-PAGE gradient gel. The gel was incubated in 1 M sodium salicylic acid, fixed, dried and exposed to X-ray film for 18 h at room temperature.

### EXAMPLE 6

Recombinant cDNA Constructs and Transfection of Cells

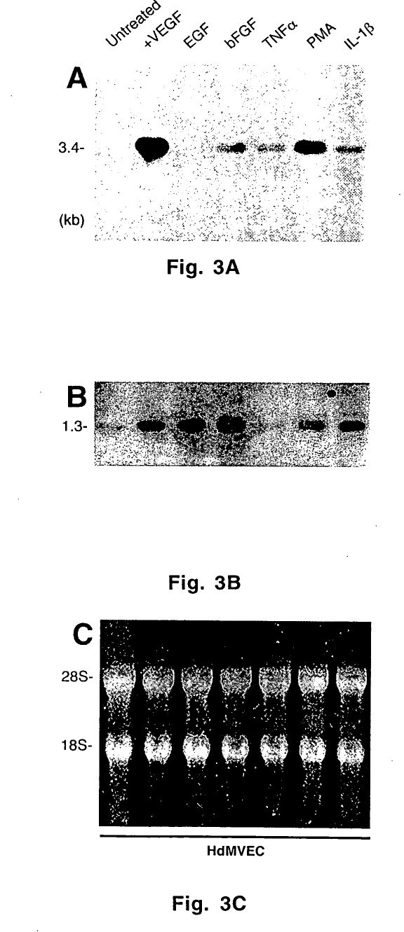

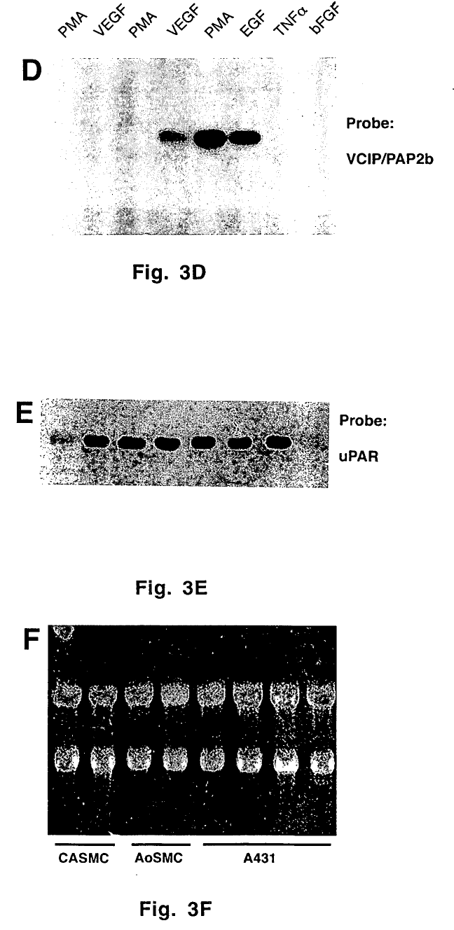

In order to generate GFP-VCIP constructs, PCR primers containing BamHI (5′) and HindIII (3′) restriction sites were designed. The GFP gene was inserted in-frame with the VCIP gene (on either the N-terminus or C-terminus) into the mammalian expression plasmids pEGFP-N3 or pEGFP-C3, thereby producing pEGFP-VCIP-N3 or pEGFP-VCIP-C3 fusion proteins (FIG. 3A-D). For retroviral constructs, human VCIP cDNA was subcloned into pLNCX2 (Clontech) immediately downstream of the human CMV immediate early promoter. Two-step PCR was used to insert three copies of an HA-tag (YPYDVPDYA, SEQ ID NO:12) at the N-terminus of the VCIP cDNA and to mutate the wild-type RGD sequence in one of the proteins to RGE (FIG. 3E-G). The two-step PCR method has been described previously (Wary et al., 1996). Amphopack-293 packaging cells (Clontech) were transfected with pLNCX2 (V), pLNCX2-VCIP-RGD (WT) and pLNCX2-VCIP-RGE (MT) using Superfect™ liposome (Qiagen). Supernatants collected from stably transfected packaging cell lines were incubated with 60% confluent HEK293 cells in the presence of polybrene (8 μg/ml). For GST-fusion proteins, two-step PCR was used to mutate the wild-type RGD sequence in one of the proteins to RGE (FIG. 3H-J). Fragments were subcloned into the BamHI and HindIII restriction sites of the pGstag vector (Ron and Dressler, 1992), and constructs were confirmed by DNA sequencing. The GST-VCIP-RGD and GST-VCIP-RGE recombinant proteins were expressed in Escherichia coli (BL21) cells and affinity purified using Sepharose-glutathione beads. GST-fusion proteins were dialyzed against 20 mM Tris-HCl pH 7.5, containing 175 mM sodium chloride and 20 mM potassium chloride.

### EXAMPLE 7

Cell Aggregation Assay

To monitor aggregation, cells were labeled with optimal non-toxic concentrations of fluorescent dyes. This assay was performed essentially according to the protocol described by Niessen and Gumbiner (2002), with minor modifications. Briefly, pLNCX2-VCIP-RGD-HEK (WT) and pLNCX2-VCIP-RGD-HEK (MT) cells were detached from dishes with 0.025% trypsin and 2 mMEDTA, washed with PBS and passed through a cell strainer. Cells were collected and resuspended in HCMF buffer (20 mM HEPES pH 7.4, 137.5 mM NaCl, 5.0 mM KCl, 0.35 mM Na₂HPO₄.7 H2O, 4.5 mM glucose and 10 mM CaCl₂) supplemented with 5 mM Ca²⁺, 1 mM Mg²⁺, 10 μg/ml of 3,3-dioctadecycloxacarbocyanine perchlorate (DiO) and 2.5 μg/ml of 1,1′-dioctadecyl-3,3,3,′3′-tetramethylindocarbocyanine perchlorate (Dil) (Molecular Probes) at 37° C. for 8 min. Red and green cells (0.5×10⁶ of each) were allowed to aggregate in 500 μl of HCMF in the presence of 50 U/ml DNase I containing either Ca/Mg, peptides or EDTA, or anti-VCIP-RGD or control antibodies in siliconized cylindrical glass vials by rotating at 90 r.p.m. at 37° C. for 0, 6 or 12 h. The inhibitory effects of EDTA, peptides and antibodies on cell aggregation were determined at the end of 12 h. A graticule was placed inside a lox eyepiece to aid enumeration of cell aggregates. A minimum of seven random fields were used for each time point. Experiments were performed at least three times with each time point analyzed in triplicate. Only productive cell aggregates (yellow) were counted. Unproductive WT (red) cell aggregates were ignored. Numbers were expressed as the percentage of total aggregates counted.

### EXAMPLE 8

Cell Proliferation, Apoptosis and Immunofluorescence Microscopy

The methods used to measure proliferation and score apoptosis have been described (Wary et al., 1996). Briefly, cells were deprived of growth factors for 24 h. The next day, cells were replenished with defined media containing 10 μM BrdU and returned to the 37° C. incubator for 16-18 h. Cells were then fixed and permeabilized by acid treatment, immunostained with an anti-BrdU monoclonal antibody and an alkaline phosphatase-conjugated secondary antibody, then counterstained with hematoxylin. The BrdU-positive cells were scored from three independent experiments performed in triplicate. A minimum of five random fields was selected on each coverslip at 100× magnification. The percentage of BrdU incorporation was determined as a measure of the number of cells entering the S phase of the cell cycle.

For the apoptosis assay, cells were deprived of growth factors for 24 h, then incubated in defined medium for 28 h. Attached and unattached cells were combined, fixed with cold 20 mM glycine-HCl pH 2.0 and stained in suspension with Hoechst 33258 dye (0.5 μg/ml). Cells were examined under a Zeiss Axiovert-125 fluoroscope. The presence of more than two visible nuclear fragments was considered as a single apoptotic event. Apoptotic events were counted from at least five random microscopic fields.

### EXAMPLE 9

Solid Phase ELISA and Adhesion Blocking Assay

Solid phase ligand binding assays were performed according to a previously published procedure (Orlando and Cheresh, 1991). Briefly, soluble α2β1, α5β1, αvβv3 and αvβ5 integrins (1 μg/ml in a solution containing 20 mM Tris pH 7.4, 150 mM NaCl, 1 mM CaCl₂, 1 mM MgCl₂ and 1 mM MnCl₂) were immobilized onto 96-well microtiter plates at 4° C. Wells were washed and blocked with 0.5% BSA. After washing, GST, GST-VCIP-RGD and GST-VCIP-RGE ligands were added (50-350 ng per well in a solution of TBS pH 7.4) and incubated at 37° C. for 1 h. After washing, the wells were incubated with the anti-GST (sc-138, Santa Cruz) monoclonal antibody for 1 h, followed by washing and incubation with a horseradish peroxidase (HRP)-conjugated mouse secondary antibody. Plates were then washed again and the ABTS substrate (Bio-Rad) was added. All washing steps were carried out using PBS. Absorbances were read at 405 nm, and non-specific binding values were adjusted against BSA.

For adhesion blocking assays, endothelial cells were detached, washed with PBS and resuspended in M199 media containing 1 mM CaCl₂ and 1 mM MgCl₂ in the absence of serum or growth factors. Endothelial cells (2×10⁵ cells) were replated onto 24-well tissue culture plates coated with 1, 5 or 10 nM affinity purified GST-VCIP-RGE or GST-VCIP-RGD fusion proteins. Cells were allowed to reattach for 45 min, then washed, fixed with 4% paraformaldehyde, stained with 0.5% crystal violet for 5 min and then washed extensively with water. Absorbances were measured at 540 nm.

To monitor the effects of anti-integrin antibodies, dishes were coated with 10 nM GST-VCIP-RGD, immobilized with 1.0% glutaraldehyde in PBS and washed several times with PBS prior to use. Endothelial cells (5×10⁵ cells in 300 μl PBS) were pre-incubated at 4° C. with 1, 5 or 10 μg/ml of anti-α5 β1 (P1B6, Invitrogen), anti-αvβ3 (LM609, Chemicon), anti-α2β1 (MAB 1998, Chemicon) or anti-α3β1 (P1B5, Invitrogen) antibodies for 30 min. Cells were then washed with PBS containing Ca²⁺ and Mg²⁺, and replated onto coated dishes. After 45 min, cells were washed, fixed and stained with 0.05% crystal violet for 10 min. After extensive washing, absorbances of eluted dyes were measured at 590 nm.

### EXAMPLE 10

Immunostaining of Tumor Sections

Double immunostaining of paraffin-embedded tumor sections (4 μm) was performed following antigen retrieval. Specimens were subjected to microwave treatment (1000 W) in citrate buffer pH 6.0, four times for 5 min each. Peroxidase activity was then inhibited by the addition of 3% H₂O₂ in PBS for 20 min, followed by blocking with 3% BSA in PBS. Sections were then incubated with the affinity purified anti-VCIP-RGD antibody (20 μg/ml), followed by either anti-VEGF (30 μg/ml), vWF (50 μg/ml) or anti-αvβ3 integrin (30 μg/ml) antibodies. After incubation with primary antibodies, slides were washed with PBS. Incubation with Texas Red-conjugated anti-rabbit IgGs and with FITC-conjugated goat anti-mouse IgGs was used to detect VCIP (red), vWF (green) and αvβ3 (green) integrin, respectively.

### EXAMPLE 11

Cloning of PAP2b/VEGF and Type I Collagen Inducible Protein (VCIP)

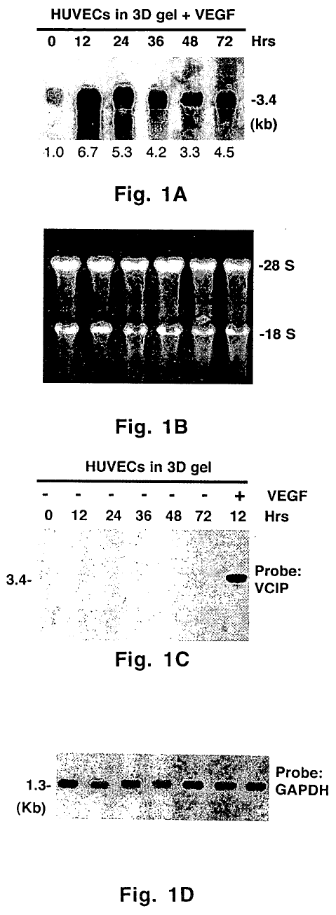

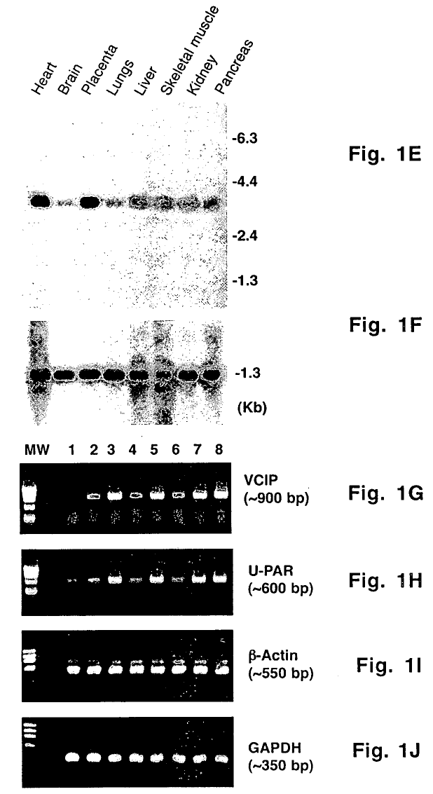

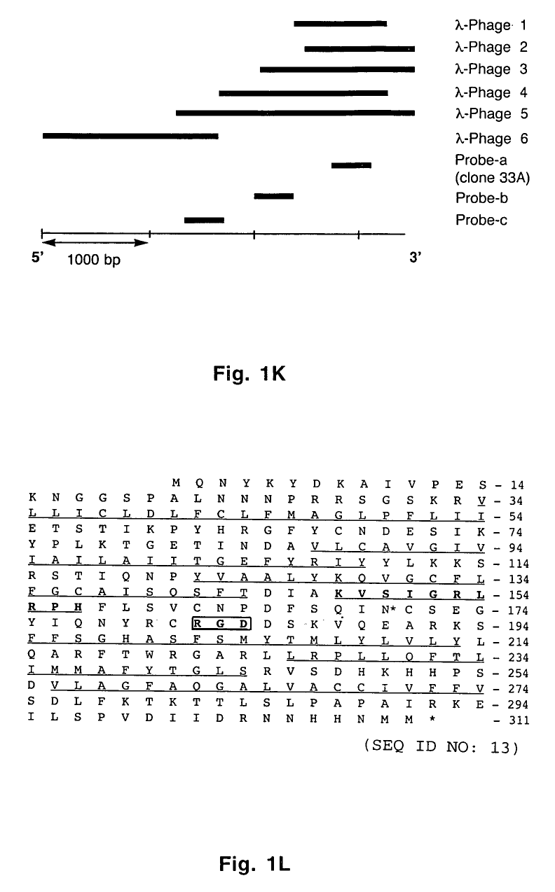

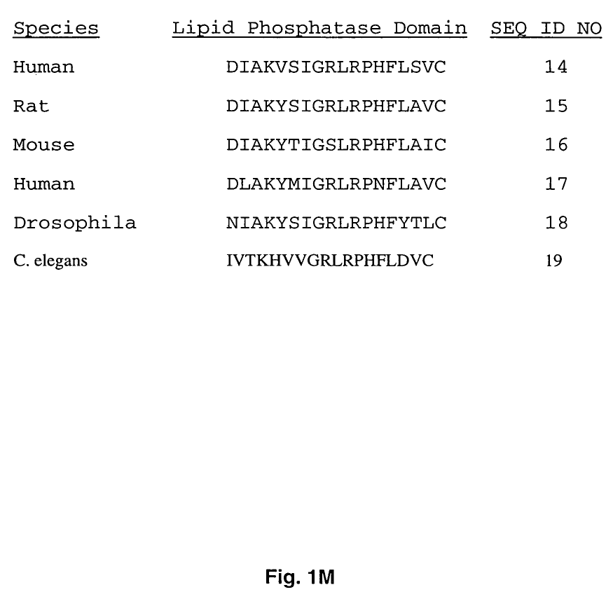

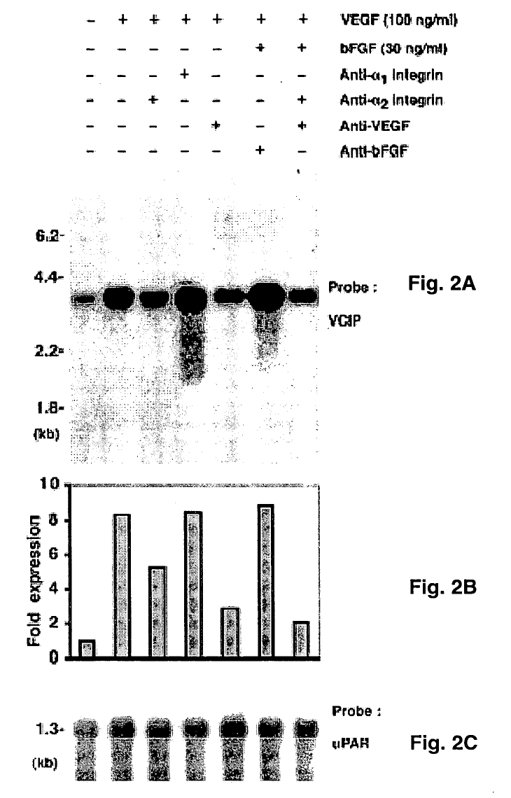

In a previous study, endothelial cells were embedded into three dimensional type I collagen gel and induced to undergo capillary morphogenesis in response to VEGF¹⁶⁵. RNA was then isolated from endothelial cells cultured in the presence or absence of VEGF¹⁶⁵, converted to cDNA and subjected to suppression subtractive hybridization and differential display. Through this process, a set of 12 candidate genes associated with capillary morphogenesis were identified in endothelial cells. One of the gene fragments (˜500 bp) identified with this approach was of particular interest. Initial northern blot analyses suggested that its expression required presence of VEGF (FIGS. 1A and C). In a subsequent study, it was found that the addition of anti-VEGF or anti-α2β1 integrin (type I collagen receptor) antibodies partially blocked expression of this gene in endothelial cells (FIG. 2). For this reason, this gene was designated VCIP (VEGF and type I collagen inducible protein).

VCIP mRNA was most strongly expressed in human heart and placenta, tissues that are highly vascularized (FIG. 1E). Next, expression of VCIP in monolayer endothelial cells treated with bFGF, VEGF and PMA was examined (FIG. 1G). It was found that all three cytokines were equally able to induce expression of VCIP. This pattern of regulation was identical to that of the human receptor for urokinase plasminogen activator (uPAR) expression (FIG. 1H). β-actin and GAPDH were included as controls and were not regulated under any of these conditions (FIGS. 1I and J).

The VCIP gene was cloned to investigate its possible role in capillary morphogenesis of endothelial cells. During initial cloning effort, the investigators sequenced several 3′ ends of RT-PCR products derived from a pool of 3′ and 5′ RACE products using DNA sequence information from clone 33A, as shown in FIG. 1K. Sequencing of a ˜2.0 kb 3′ region of a putative gene did not exhibit an open reading frame (ORF). Two 5′ probes were generated from this 2.0 kb fragment, and used to screen a human placental cDNA library, resulting in the recovery of six _-phage cDNA clones (FIG. 1K). Sequencing analysis revealed that one of the cDNA fragments of ˜1.5 kb in size contained an ORF of 930 bp (_-phage 6; FIG. 1K), which clearly corresponded to the PAP2b gene (Kai et al., 1997). In fact, the cDNA that designated as VCIP turned out to be identical to PAP2b, which encodes a 3.4 kb transcript. This is much larger than the transcript size that was previously reported for PAP2b (Kai et al., 1997). However, the data show that PAP2b/VCIP (henceforth called VCIP) has an unusually long 3′ untranslated region (UTR) of ˜2.0 kb. The complete cDNA sequence of VCIP has been deposited under the accession No. AF480883, and the deduced amino acid sequence of VCIP is shown in FIG. 1L. Database searches with the VCIP coding sequence and analysis of the deduced amino acid sequence revealed that VCIP has a consensus lipid phosphatase motif and an RGD cell attachment sequence in the second extracellular domain, whereas the cytoplasmic domains of VCIP lack any known enzymatic features or motifs. The lipid phosphatase motif of VCIP is shown in comparison with other known lipid phosphatase motifs in FIG. 1M.

### EXAMPLE 12

Growth Factors and Inflammatory Cytokines Induce Expression of VCIP

To examine VCIP expression by other cell types, endothelial, smooth muscle and epithelial cells (A431) were stimulated in monolayer with various growth factors and cytokines for 6 h. Total RNA was then isolated and subjected to northern blot analysis for VCIP and uPAR (FIG. 3). Human dermal microvascular endothelial cells (HdMVECs) responded to most cytokines, but not to EGF. VCIP was expressed most strongly in cells that were stimulated with VEGF (FIG. 3A, lane 2). Neither PMA nor VEGF¹⁶⁵ induced expression of VCIP in carotid artery smooth muscle cells (CASMCs), whereas VEGF¹⁶⁵ increased VCIP levels in aortic smooth muscle cells (AoSMC), but PMA did not. VCIP levels were strongly induced by PMA and EGF in epidermoid carcinoma (A431) cells, whereas TNF-α and bFGF had no effect in this cell type.

### EXAMPLE 13

VCIP is a Cell Surface Protein

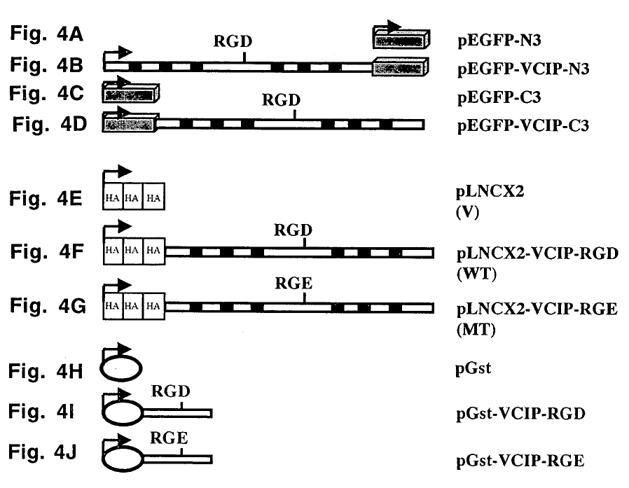

Using confocal microscopy, Ishikawa et al. (2000) showed that PAP2b is localized at the plasma membrane in transfected cells. To determine whether VCIP is a plasma membrane protein that is exposed on the cell surface, HEK293 cells were transfected with green fluorescent protein (GFP)-VCIP fusion proteins. A diagram of the constructs is shown in FIG. 4.

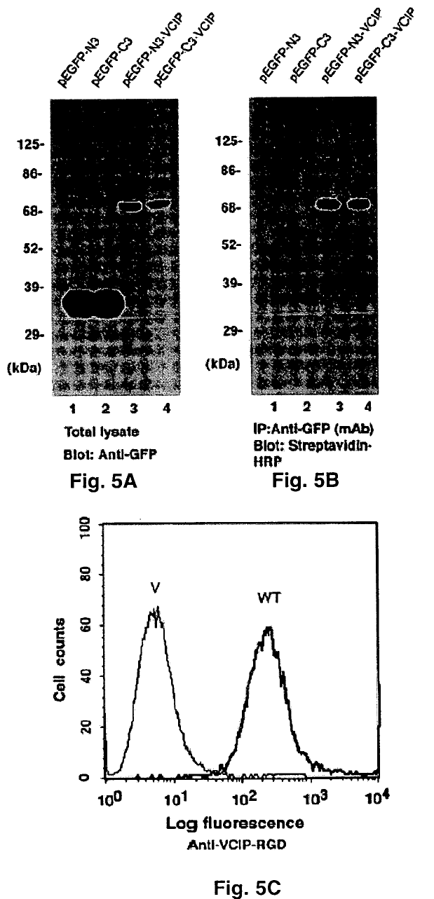

Transfected cells were detached from culture dishes and subjected to cell surface biotinylation. Proteins were subjected to immunoblotting or immunoprecipitation with anti-GFP antibodies. Cells transfected with the control GFP vector exhibited a ˜30 kDa GFP-immunoreactive band (FIG. 5A, lanes 1 and 2), whereas a GFP-immunoreactive band of ˜68 kDa was detected in lysates from cells transfected with the GFP-VCIP expression vector (FIG. 5B, lanes 3 and 4). No biotinylated proteins were detected in anti-GFP immunoprecipitates from HEK293 transfected with vector alone (FIG. 5B, lanes 1 and 2), whereas the anti-GFP antibody immunoprecipitated a ˜68 kDa biotinylated polypeptide from cells transfected with the pEGFPVCIP-N3 or -C3 expression vectors (FIG. 5B, lanes 3 and 4). Subsequent immunoprecipitation and immunoblot analysis of cell lysates obtained from HUVECs and HdMVECs showed that the Mr of non-glycosylated VCIP is ˜38 kDa and that N-glycosylated VCIP is 44/48 kDa. Incubation of the VCIP antigen-antibody complex with N-glycanase removed the carbohydrate moiety, and the glycosylated forms of this protein were not detectable (data not shown). Furthermore, as shown in FIG. 5C, fluorescence-activated cell sorting (FACS) analysis showed that the VCIP-RGD sequence is located outside of the cell surface of 293HEK cells.

### EXAMPLE 14

Retroviral Transduction of VCIP Promotes Cell-Cell Interactions

The effects of wild-type VCIP (RGD) versus mutant VCIP (RGE) were evaluated in a cell system that allowed study of the role of the VCIP-RGD sequence. Primary endothelial cells were not considered suitable for generating stable clones, therefore, HEK293 cells were used to create stable cell lines. HEK293 cells were chosen because they are easily transfected and do not express endogenous VCIP protein. cDNA constructs encoding the various retroviral VCIP constructs were generated (FIG. 4E-G). Several clones of HEK293 cells stably expressing wild-type VCIP (pLNCX2-VCIP-RGD-HEK, WT), mutant VCIP (pLNCX2-VCIP-RGE-HEK, MT), or vector alone (pLNCX2-HEK, V) were obtained under geneticin (G418) selection. Three independent clones were isolated for each construct to ensure that any observed effects were not due to phenotypic variability intrinsic to cultured cells. Clones were regularly evaluated by semi-quantitative RT-PCR (20 cycles) and by immunoblotting to ensure that mRNA and protein expression levels were not altered (data not shown). pLNCX2-HEK (V) was chosen as a control because it is has no known effect on cells.

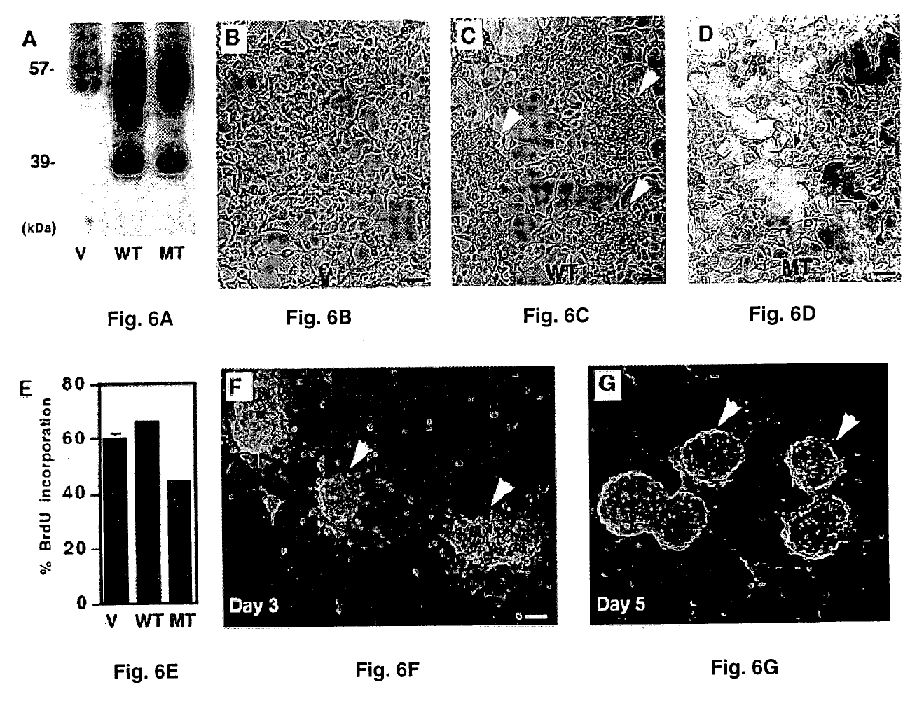

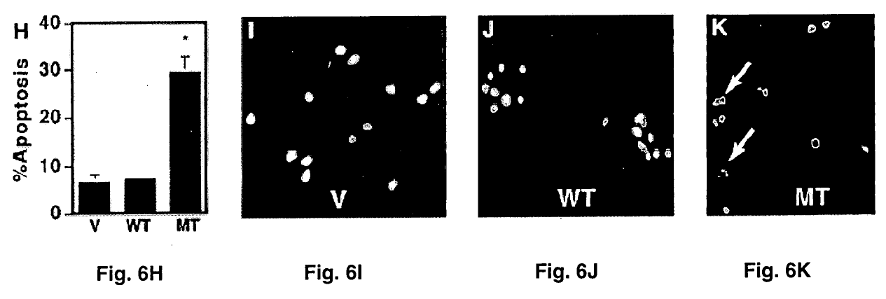

Cell lysates were subjected to immunoprecipitation with an anti-VCIP-cyto antibody, and analyzed by anti-hemaglutinin (HA) immunoblotting. WT and MT cells expressed equivalent levels of VCIP immunoreactivity (FIG. 6A). It was observed that WT cells formed cell-cell contacts (cell aggregates), whereas MT and V cells did not (FIGS. 6B-D). The rate of formation of cell aggregates in WT cells was dependent on the number of cells seeded. When 2×10⁵ cells were seeded sparsely in a 35 mm dish, the formation of cell aggregates was relatively slow (data not shown). In fact, when WT cells were seeded sparsely, they underwent at least one cell division prior to forming such cell aggregates. The formation of cell aggregates accelerated when 5×10⁷ cells were initially seeded (data not shown). As the time in culture progressed, cell aggregates of various sizes were visually recognizable within 24-36 h of seeding (FIG. 6C). Cell aggregates continued to grow in size until 98 h or longer. As the colonies increased in size, most of the surrounding cells migrated (relocated) and adhered to the growing cell mass. Eventually, the colonies grew to a sufficiently large size so that they detached from the dishes.

WT cells were also incubated with several peptides modeled after the VCIP-RGD region. When WT cells were cultured in the continuous presence of an anti-VCIP-RGD antibody (25-50 μl/ml) and NYRCRGDDSK (SEQ ID NO:20) (10-50 nM), the size, the speed of formation and the number of such cell aggregates were reduced (Table 1). In contrast, no reduction in cell aggregation was observed in cells incubated with the mutant peptides NYRCRADDSK (SEQ ID NO:21) (10-50 nM) or NYRCRGEDSK (SEQ ID NO:22) (10-50 nM). Incubation with the antibody or peptides did not induce toxicity or cell death. The cell aggregation observed in WT cells was specific, in that cells transfected with pLNCX2-HEK or pLNCX2-VCIP-RGE-HEK did not exhibit such phenotype (FIGS. 6B and D).

In order to eliminate the possibility of clonal variation, three independent clones of WT cells were examined. This phenotype was also reproduced in NIH 3T3 cells under similar experimental conditions. High resolution photomicrographs of living cell cultures demonstrated the progressive formation of cell aggregates by WT cells at days 3 and 5, as shown in FIGS. 6F-G. In addition, three different clones of WT cells embedded in soft agar supplemented with complete media failed to show anchorage-independent cell growth or colony formation.

Next, the ability of VCIP proteins to regulate proliferation and apoptosis in HEK293 cells was examined. As shown in FIG. 6E, WT cells were clearly proliferation competent, as VCIP expression increased the number of BrdU-positive cells by ˜65%, which was comparable to the number of BrdU-positive cells in control V cells (˜60%). In parallel, cells were incubated in defined media and apoptosis was evaluated after 36 h (FIG. 6H). Interestingly, MT cells showed the highest levels of apoptosis (˜25% of all nuclei were apoptotic), whereas V and WT cells exhibited only baseline levels of apoptosis (˜6±8%). Representative photomicrographs of Hoechst-stained cells are shown in FIG. 6I-K.

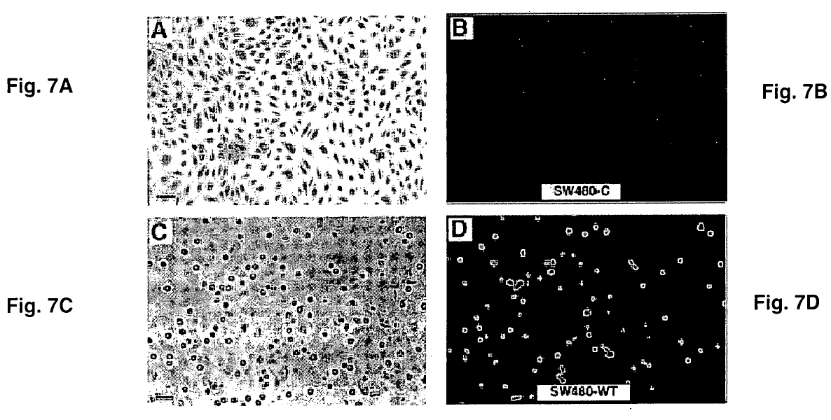

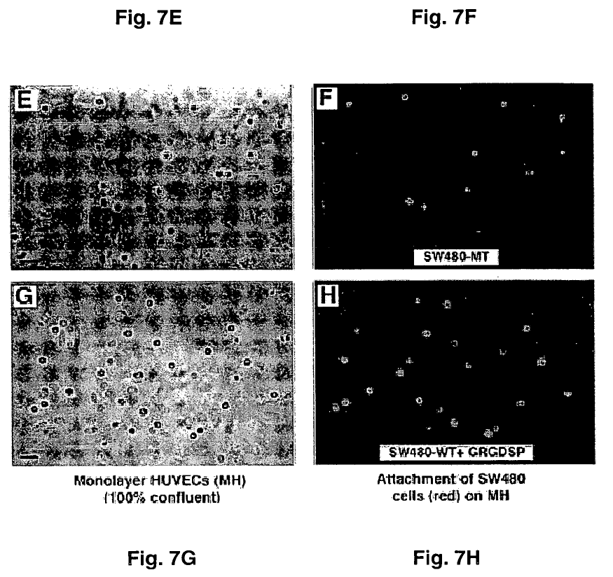

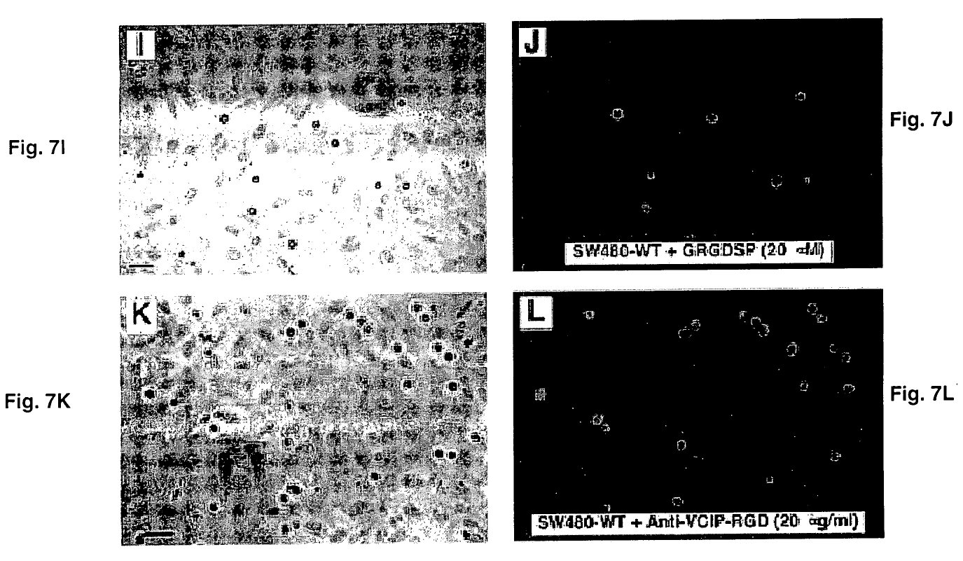

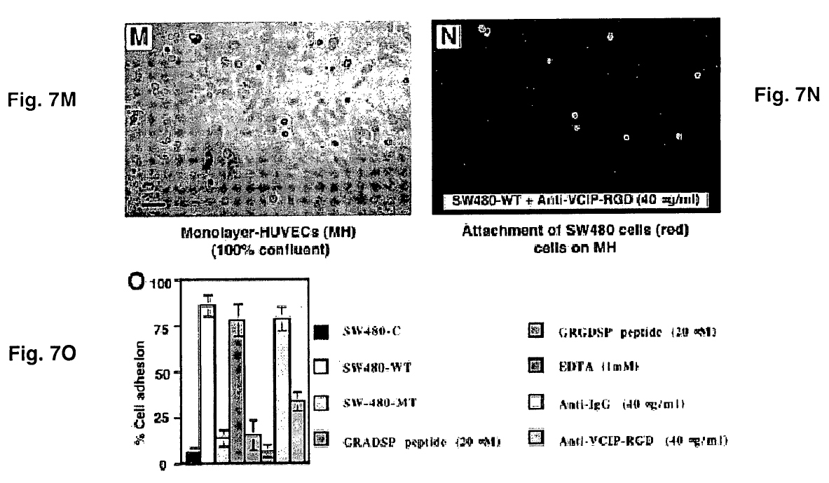

In addition, cadherin-deficient SW480 cells stably expressing the pLNCX2-VCIP-RGD construct attached to monolayer HUVECs, whereas cells expressing pLNCX2 or pLNCX2-VCIP-RGE did not. Adhesion of pLNCX2-VCIP-RGDSW480 cells to monolayer HUVECs was blocked by incubation with the anti-VCIP-RGD antibody and the GRGDSP (SEQ ID NO:11) peptide in a dose-dependent manner (FIG. 7), whereas control substances had no significant effect on adhesion.

TABLE 1
Cell Aggregation

| HEK cells stably expressing | Substance | Concentration | % cell aggregates |
|---|---|---|---|
| pLNCX2 (vector alone) (V) | | | 0 |
| pLNCX2-VCIP-RGD (WT) | | | 94 ± 5.5^{a} |
| pLNCX2-VCIP-RGE (MT) | | | 0 |
| pLNCX2-VCIP-RGD (WT) | Anti-rabbit IgG | 25 μg/ml | 90 ± 7.3 |
| pLNCX2-VCIP-RGD (WT) | Anti-rabbit IgG | 50 μg/ml | 91 ± 8.8 |
| pLNCX2-VCIP-RGD (WT) | Anti-VCIP-RGD | 25 μg/ml | 73 ± 7.4 |
| pLNCX2-VCIP-RGD (WT) | Anti-VCIP-RGD | 50 μg/ml | 36 ± 12.5^{a} |
| pLNCX2-VCIP-RGD (WT) | NYRCRGDDSK | 10 nM | 67 ± 15.7 |
| pLNCX2-VCIP-RGD (WT) | NYRCRGDDSK | 30 nM | 46 ± 13.3 |
| pLNCX2-VCIP-RGD (WT) | NYRCRGDDSK | 50 nM | 28 ± 10.5^{a} |
| pLNCX2-VCIP-RGD (WT) | NYRCRADDSK | 10 nM | 92 ± 5.7 |
| pLNCX2-VCIP-RGD (WT) | NYRCRADDSK | 30 nM | 89 ± 7.5 |
| pLNCX2-VCIP-RGD (WT) | NYRCRADDSK | 50 nM | 90 ± 7.2^{a} |
| 2.0 × 10⁵ HEK cells were pre-treated with indicated substance, washed with PBS, plated in defined media and allowed to form cell aggregates at 37° C. This experiment was carried out using 12-well tissue culture plates. Fresh aliquots of substances were added every 12 h. The number of cell aggregates formed was enumerated at the end of 48 h. Typically, 8-12 cell aggregates were visible in a single 100x microscopic field. | | | |
| # At least 5-7 random fields were selected. Experiments were performed three times in triplicate. Values are mean ± SD. ^{a}P < 0.05. | | | |

### EXAMPLE 15

VCIP-Mediated Signaling

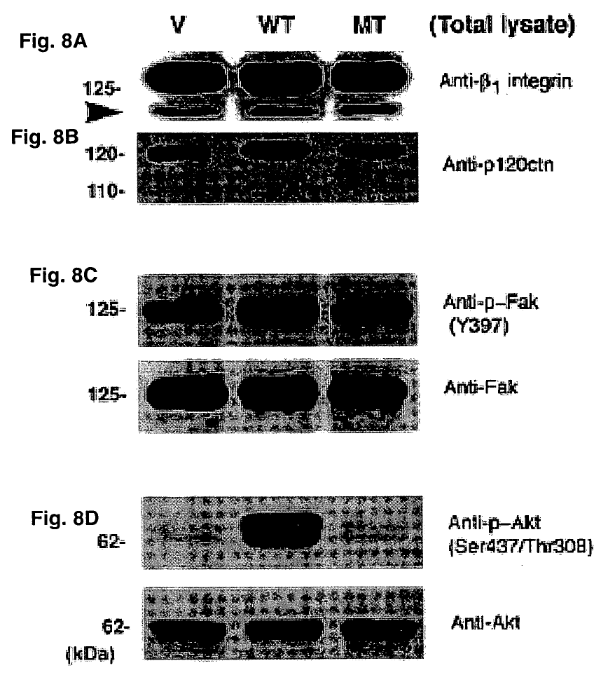

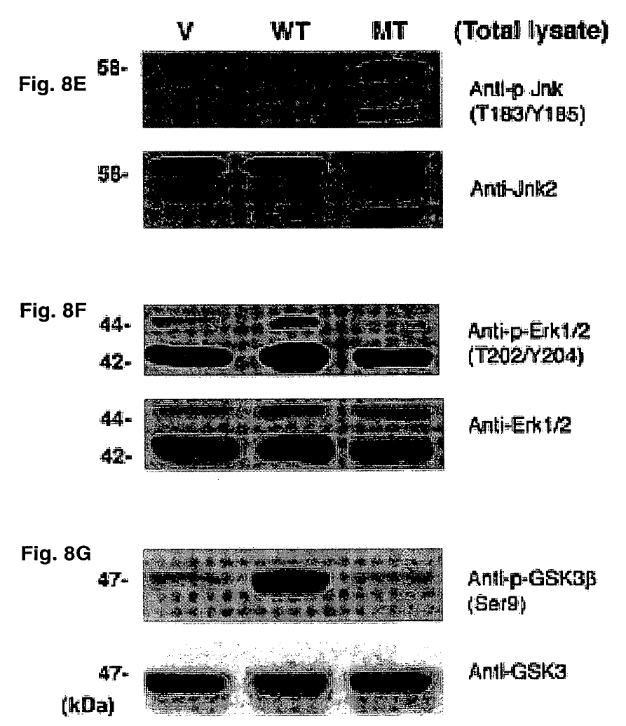

Next, the question of whether expression of VCIP influences the growth properties of HEK293 cells was addressed. To identify the molecular events associated with pLNCX2-VCIP-RGD-HEK cell-cell interactions, β1 integrin and p120catenin (p120ctn) protein levels were measured. The phosphorylation state and total protein levels of Fak, Akt, GSK3β and Erk2 protein kinases, which play roles in adhesion-mediated cell proliferation, survival and migration were also measured. Enzymatic activation of these protein kinases is accompanied by an increase in phosphorylation state. β1 integrin immunoreactivity levels were similar in V, WT and MT cells (FIG. 8A). Interestingly, p120ctn immunoreactivity levels were increased in WT cells, as compared with V or MT cells. Similarly, the phosphorylation state of Fak, Akt and GSK3β were increased in WT cells, as compared with V or MT cells (FIGS. 8C, D and G). The phosphorylation state of Erk½ was only modestly increased in WT cells, as compared with the dramatic increase in phosphorylation state of Akt in WT cells (FIGS. 8D and F).

In contrast, the phosphorylation state of Jnk was not different in WT and V cells, but was moderately increased in MT cells (FIG. 8E). In MT cells, a relative decrease in the phosphorylation of Akt (FIG. 8D), together with a moderate increase in the phosphorylation state of Jnk (FIG. 8E), may contribute to the higher levels of apoptosis observed in these cells (FIG. 6H). There were no differences in the total levels of Fak, Akt, Jnk, Erk ½ or GSK3β proteins in V, WT and MT cells. Similarly, there was no difference in the level of expression of β-catenin, γ-catenin and pancadherin as determined by western blotting analyses in any of these cells (data not shown).

### EXAMPLE 16

VCIP Promotes Direct Cell-Cell Interactions

Because expression of wild-type, but not mutant VCIP induced spontaneous ‘cell-cell interactions’ (cell aggregation) in 293HEK cells, VCIP-RGD could act as a cell-associated integrin ligand. Thereby, VCIP-RGD could promote ‘cell±cell interactions’ by specifically recognizing αvβ3 and α5β1 integrins presented on adjacent cells.

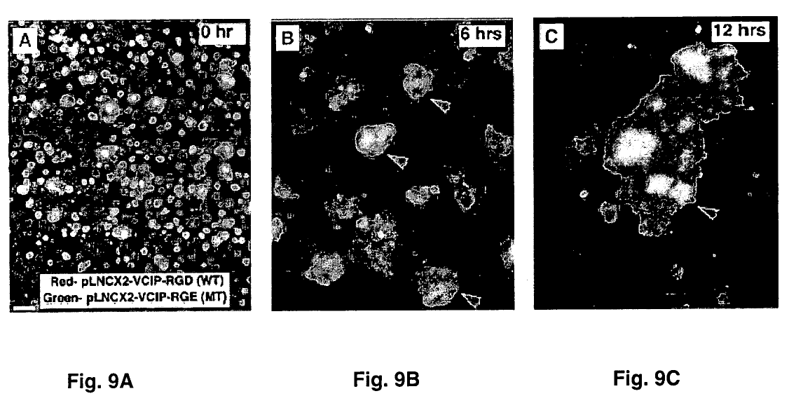

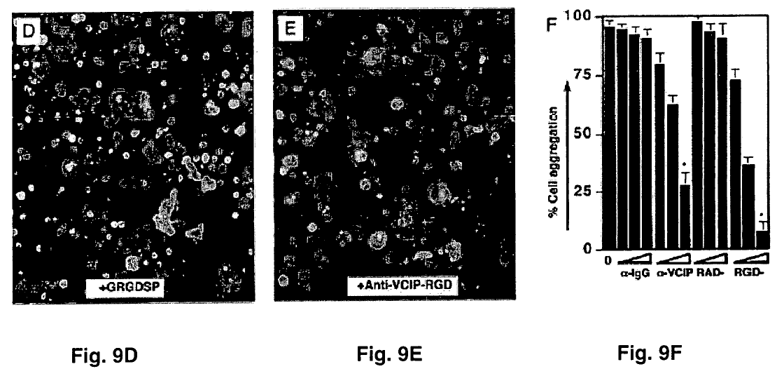

As shown in FIG. 9, the mixing of WT cells with MT cells (0.5×10⁶) resulted in the formation of at least 10-15 small and large aggregates within 6 h. These interactions were effectively inhibited by pre-incubating WT cells with an anti-VCIP-RGD antibody, but not with control antibodies. Similar results were obtained when WT cells were incubated with the GRGDSP (SEQ ID NO:11) (25 μM) peptide or the anti-VCIP-RGD antibody (FIGS. 8D and E). Dose-dependent inhibition of cell aggregates in response to the GRGDSP (SEQ ID NO:11) peptide and anti-VCIP-RGD antibody are shown in FIG. 9F.

Since 293HEK cells express high level of α5β1, but somewhat relatively low in αvβ3 integrin heterodimer, WT cells were mixed with cells expressing high levels of the β3 integrin subunit to evaluate the effects on cell aggregation. 293HEK cells were stably transfected with the wild-type human β3 integrin subunit. Expression levels were determined by FACS and western analyses. Mixing of WT cells with β3 integrin-293HEK cells quickly resulted in significant cell aggregation within 3-6 h (data not shown).

### EXAMPLE 17

VCIP Interacts with αvβ3 And α5β1 Integrins

In view of the above findings, whether recombinant VCIP expression could promote adhesion of endothelial cells in primary culture was examined. In order to determine whether the VCIP-RGD motif acts as an integrin ligand, two recombinant VCIP fragments (each 49 amino acids in length) that corresponded to a predicted second extracellular loop of the protein were generated (FIGS. 1L and 3H-J). The recombinant GST-VCIP-RGD protein is composed of 49 amino acid residues (amino acid residues 145-194, FIG. 1L). It contains the lipid phosphatase motif (KXXXXXXRP), but lacks a proton donor sequence, i.e., PSGH motif (residue 196-199, FIG. 1L) and -(X31-54)-SRXXXXXHXXXD sequence.

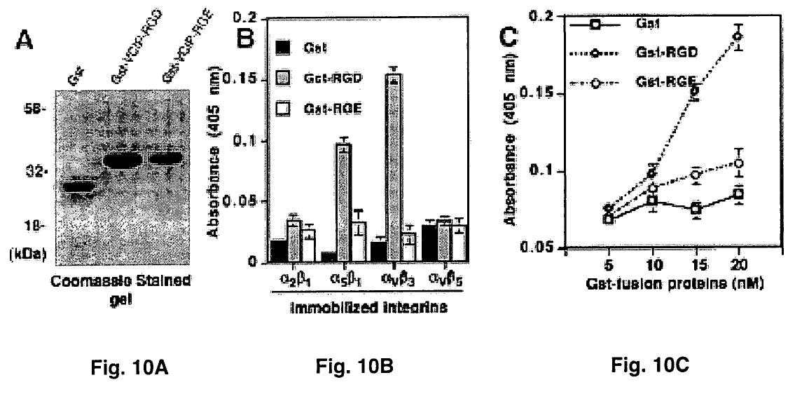

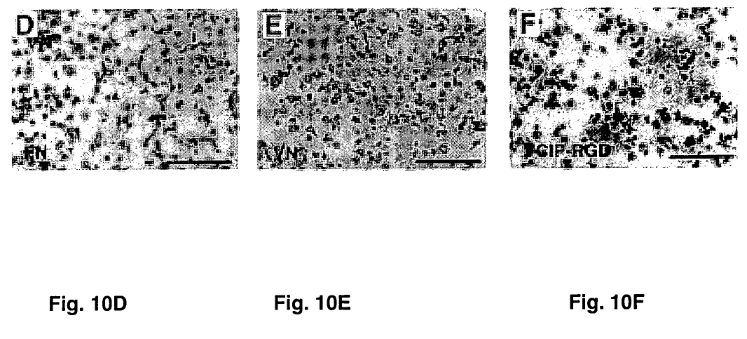

Wild-type glutathione S-transferase (GST)-VCIP-RGD and mutant GST-VCIP-RGE fusion proteins were affinity purified and visualized by Coomassie Blue staining on SDS-PAGE as shown in FIG. 10A. Both the wild-type and mutant proteins migrated at the predicted size of 34 kDa.

To determine whether GST-VCIP-RGD could interact with various integrins, a solid-phase ligand binding assay was performed. As shown in FIG. 10B, the α5β1 and αvβ3 integrin heterodimers interacted with GST-VCIP-RGD in solution with comparable affinities, whereas the α2β1 and αvβ5 integrins did not. None of these integrins significantly interacted with GST alone or with the GST-VCIP-RGE mutant, suggesting that the interaction of VCIP-RGD with α5β1 and αvβ3 integrins is highly specific. VCIP-RGD bound to αvβ3 integrin in a dose-dependent manner, whereas GST alone or GST-VCIP-RGE exhibited negligible binding to αvβ3 integrin (FIG. 10C). Representative photomicrographs of optimal adhesion and spreading of endothelial cells plated for 45 min on fibronectin, vitronectin and GST-VCIP-RGD are shown in FIG. 10D-F. Adhesion of endothelial cells to GST-VCIP-RGD was comparable to that observed in wells coated with vitronectin and fibronectin substrates.

Next, the capacity of VCIP-RGD to bind endothelial cell integrins was evaluated by determining the effect of GST-VCIP fusion proteins on cell adhesion and spreading. Endothelial cells adhered to wells coated with recombinant wild-type GST-VCIP-RGD protein in a dose-dependent manner. In contrast, there was little adhesion to wells coated with the mutant (GST-VCIP-RGE) fusion protein (FIG. 10G). Active protein synthesis was not required for endothelial cells to attach to the substrates tested, because pretreatment of endothelial cells with cycloheximide (20 μg/ml) for 1 h prior to replating the cells onto substrate-coated dishes followed by continued exposure to cycloheximide during the entire assay period did not decrease the total number of attached cells (data not shown).

Next, which integrin(s) actually mediated adhesion to the VCIP-RGD sequence was determined. To do so, endothelial cells were non-enzymatically detached from dishes, washed, pre-incubated with various blocking antibodies and washed again to remove unbound antibodies. Endothelial cells were resuspended in serum-free M199 media and immediately replated onto GST-VCIP-RGD-coated wells. Preincubation of endothelial cells with anti-α5β1 (P1D6) and anti-αvβ3 (LM609) antibodies inhibited the attachment of endothelial cells in a dose-dependent manner (FIG. 10G). Control anti-α2β1 (MAB 1998) and anti-α3β1 (P1B5) integrin-function blocking antibodies did not have any effect on adhesion of endothelial cells to GST-VCIP-RGD. When endothelial cells were co-incubated with a mixture of P1D6 (10 μg/ml) and LM609 (10 μg/ml), the cells remained rounded, indicating that adhesion of these cells to GST-VCIP-RGD was completely inhibited. In contrast, when applied alone, neither of these two antibodies completely inhibited the adhesion of endothelial cells (data not shown). It is possible that αvβ1, αvβ5 and αvβ6 integrins may also mediate the interaction between endothelial cells and VCIP-RGD to some extent. Furthermore, adhesion of endothelial cells to VCIP required the presence of Ca²⁺ and Mg²⁺, as the addition of 2.5 mM EDTA (pH 7.4) for 5 min caused the cells to detach from tissue culture dishes. In addition, when the acetylated NYRCRGDDSKVQE (VCIP-RGD) (SEQ ID NO:23) peptide was incubated with attached cells (i.e. cells that were plated on VCIP-RGD-coated wells) at a concentration of 5 nM, the cells rounded up within 5 min and eventually completely detached from the wells.

### EXAMPLE 18

Recombinant VCIP Interacts Directly with α5β1 and αvβ3 Integrins

To confirm that VCIP-RGD interacts with integrins, clarified cell lysates were obtained from [³⁵S]Met/Cys labeled HUVECs and subjected to affinity chromatography. Lysates were pre-adsorbed twice with GST-Sepharose beads to remove proteins that interact with GST-Sepharose beads non-specifically. Pre-adsorbed lysates were incubated with GST-VCIP-RGD fusion proteins (10 μg per 3 mg lysate) in the presence or absence of 25 μM GRGDSP (SEQ ID NO:11). The beads were then extensively washed. To determine whether integrins were present in the GST-VCIP-RGD pull-down complex, the contents of a tube that did not receive the GRGDSP (SEQ ID NO:11) peptide was boiled in a dissociation buffer containing 0.5% SDS. The samples were equally divided into three tubes and diluted with cold immunoprecipitation dilution buffer to adjust the concentration of SDS to <0.1%. The samples were then immediately subjected to immunoprecipitation with indicated antibodies (FIG. 10H). The results showed that the GST-VCIP-RGD pull-down complex indeed contained anti-αvβ3 and -α5β1 immunoreactivities (FIG. 10H, lanes 4 and 5).

### EXAMPLE 19

Adhesion of Endothelial Cells Through VCIP-RGD Induces Integrin-Mediated Signaling

Adhesion of cells to extracellular matrix proteins promotes clustering of integrins at the plane of the plasma membrane. In addition to promoting structural support, this event nucleates formation of a complex of signaling-competent intracellular proteins. To investigate whether adhesion of cells to VCIP-RGD results in tyrosine phosphorylation of key focal adhesion signaling proteins, p125FAK, p46/52Shc, p130Cas and paxillin were immunoprecipitated and subjected to immunoblotting with various phospho-specific antibodies.

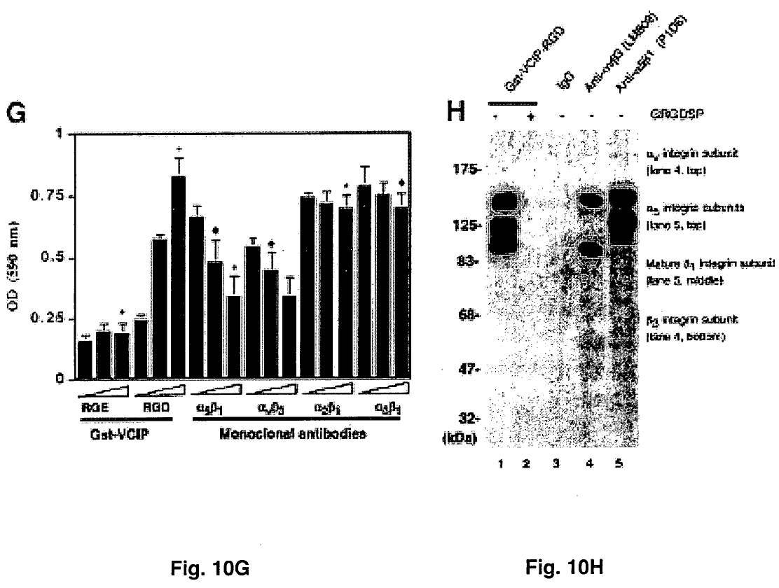

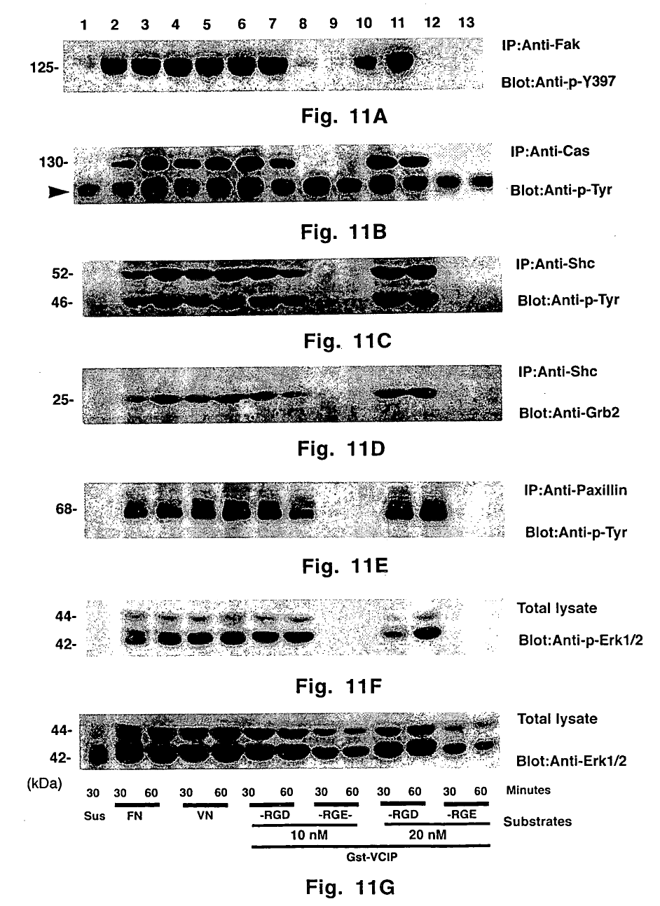

Serum- and growth factor-starved HUVECs were allowed to attach and spread on dishes coated with optimal concentrations of fibronectin, vitronectin, GST-RGD-VCIP and GST-VCIP-RGE. Cells were harvested after 30 and 60 min at 37° C. Cells were then solubilized, clarified, pre-adsorbed, immunoprecipitated and subjected to immunoblotting with various antibodies as shown in FIG. 11. VCIP-RGD induced tyrosine phosphorylation of Fak, Cas, Shc and paxillin at both 30 and 60 min. The signal intensities induced by expression of GST-VCIP-RGD were comparable to those induced by fibronectin and vitronectin. In contrast, GST-VCIP-RGE did not induce detectable tyrosine phosphorylation of Fak, Cas, Shc or paxillin. Cells that were replated onto GST-VCIP-RGE appeared rounded. Stripping and reprobing blots with anti-p125FAK, anti-p130Cas, anti-Shc and anti-paxillin antibodies showed that equal amounts of these proteins were present under all experimental conditions (data not shown).

### EXAMPLE 20

Co-Expression of VCIP With vWF And αvβ3 Integrin in Tumor Vasculature

Angiogenesis is required for the growth and survival of all solid tumors. To determine whether VCIP was expressed and co-localized with known angiogenic markers in tumor vasculatures, tumor sections were immunostained with an anti-VCIP-RGD antibody. The specificity of affinity purified anti-VCIP-RGD was confirmed by ELISA, western immunoblotting and immunolabeling experiments. Anti-VCIP-RGD reacted specifically with the GST-VCIP-RGD fusion protein, but did not react with GST-VCIP-RGE or GST alone. Moreover, the anti-VCIP-RGD antibody did not react with other RGD-containing extracellular matrix molecules such as fibronectin, vitronectin, or type I collagen.

Because the antibody did not cross-react with mouse antigens, human tissue sections were chosen for analysis. Tissue sections were initially examined by immunostaining with anti-platelet endothelial cell adhesion molecule-1 (PECAM-1, also known as CD31), anti-VE (vascular endothelial)-cadherin and anti-von Willebrand Factor (vWF) antibodies to establish the presence of endothelium. Paraffin-embedded tumor tissue sections that lacked blood vessels did not exhibit VCIP immunoreactivity. Therefore, tumor tissue sections that clearly contained endothelial cells were used. To examine whether VCIP was expressed in angiogenic tissues, serial sections of skin melanoma, angioma and normal skin tissues were examined. Enriched expressions of VEGF and αvβ3 integrin are common in angiogenic tissues, and are associated with invasion and growth of solid tumors. An increase in the levels of vWF expression is considered to be a negative prognostic factor for tumor-induced angiogenesis.

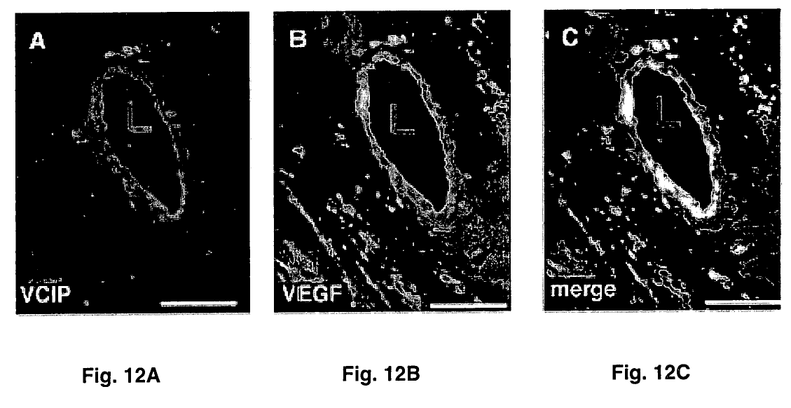

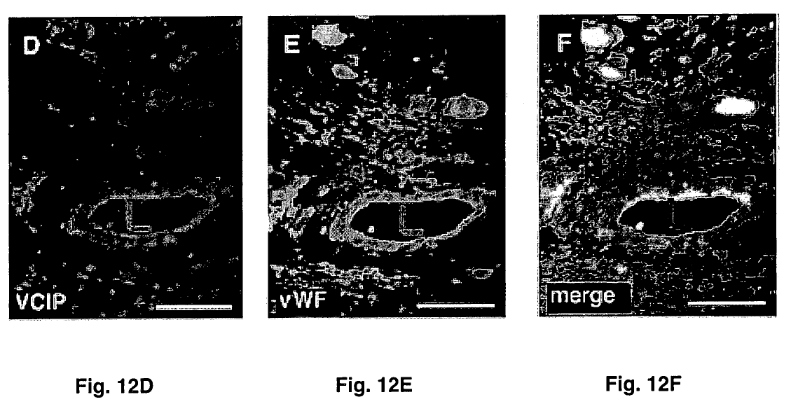

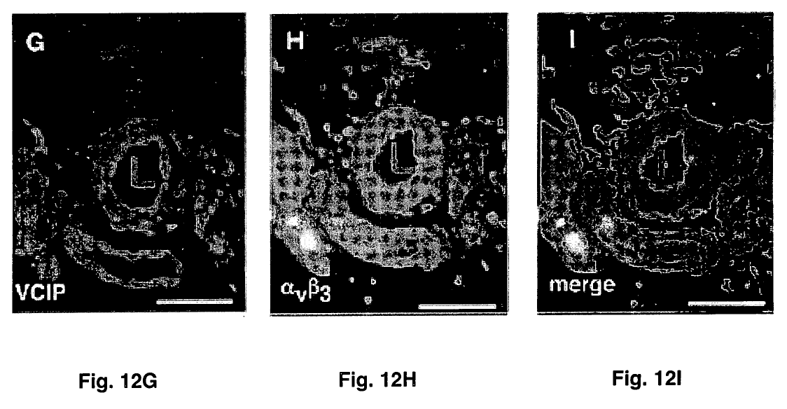

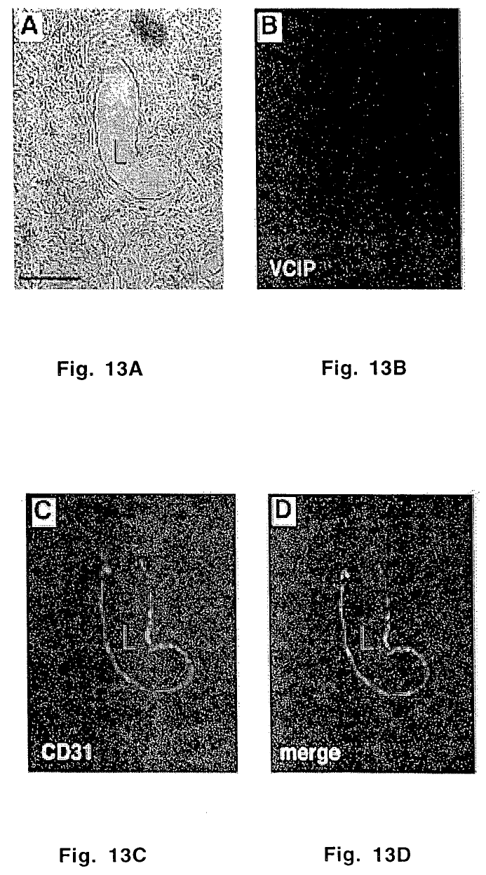

Indirect double-immunolabeling experiments showed that VCIP co-localized with vWF and VEGF in vasculatures of skin melanoma tumors (FIG. 12). VCIP also clearly co-localized with αvβ3 integrin in the angioma tissue sections examined (FIG. 12). Normal skin exhibited PECAM-1 (CD31) immunoreactivity, but not VCIP immunoreactivity (FIG. 13). VCIP immunoreactivity was lost when the affinity purified anti-VCIP antibody was incubated with the peptide used to generate the primary antibody, thereby confirming the specificity of this antibody.

### EXAMPLE 21

Overexpression of PAP2b/VCIP Impedes Endothelial Cell Migration

Activated endothelial cells display a highly motile phenotype. This motile behavior of endothelial cells is largely mediated by integrins, and it is considered to be a crucial event for angiogenesis. Sprouting of new blood vessels requires cell division in preformed endothelial tissues, such as the wall of a blood vessel, and this proliferation is accompanied by robust endothelial cells migration. Regulatory mechanisms must exist to counter migratory activity of endothelial cells, so that unnecessary (or unwanted) angiogenesis can be prevented. In view of the above results that showed VCIP-RGD serves as a cell associated integrin ligand, the present example evaluates the effect of elevated expression of VCIP on endothelial cells motility.

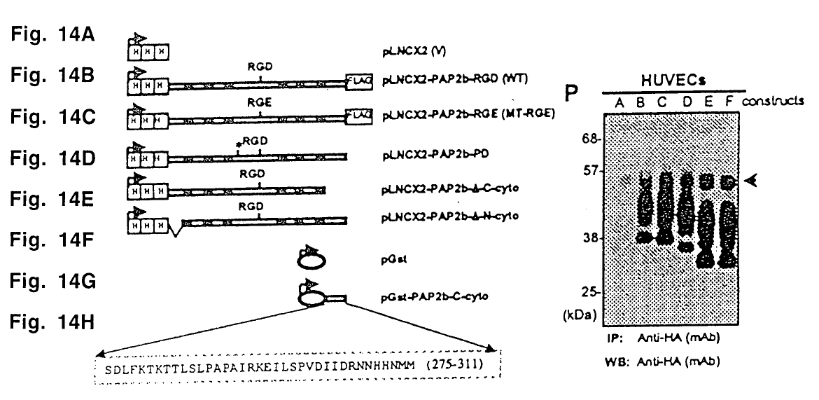

Schematics of retroviral constructs used in this and the following examples are illustrated in FIGS. 14A-H. Retroviral vector (pLNCX2) and amphotropic packaging cell line (293HEK) were bought from BD Biosciences (CA, USA). Preparation of recombinant cDNA constructs for pLNCX2-PAP2b-WT and -MT has been described previously (Humtsoe et al., 2003). Additional constructs were generated using the existing pLNCX2-PAP2b-WT as template with restriction ends BamH I and Cla I. A phosphatase inactive or dead (PD) form of PAP2b was generated by double mutation of K148A and R155A by two-step PCR strategy. The primers used were, forward: 5′-GCCGGATCCATGCAAAACTACAAGTACGAC-3′ (SEQ ID NO:24) and reverse: 5′-GAGGAGCCAGGCGCCCTATGGACACTGCGGCAAT-3′ (SEQ ID NO:25); forward: 5′-TGCCGCAGTGTCCATAGGbCGCCTGGCTCCTCA-3′ (SEQ ID NO:26) and reverse: 5′-GCGATCGATCTACATCATGTTGTG-3′ (SEQ ID NO:27). An N-terminal (_-N-cyto-1-33) and C-terminal (_-C-Cyto-284-311) PAP2b truncation was generated using primers, forward: 5′-GCCGGATCCATGCAAAAGCGGGTGCTG-3′ (SEQ ID NO:28) and reverse: GGTATCGATAAGCTTCTACATCATG-3′ (SEQ ID NO:29); forward: 5′-GCCGGATCCATGCAAAACTACAAGTACGAC-3′ (SEQ ID NO:30) and reverse: 5′-CGCGATCGATCTACGTCGTCTTAGT-3′ (SEQ ID NO:31), respectively.

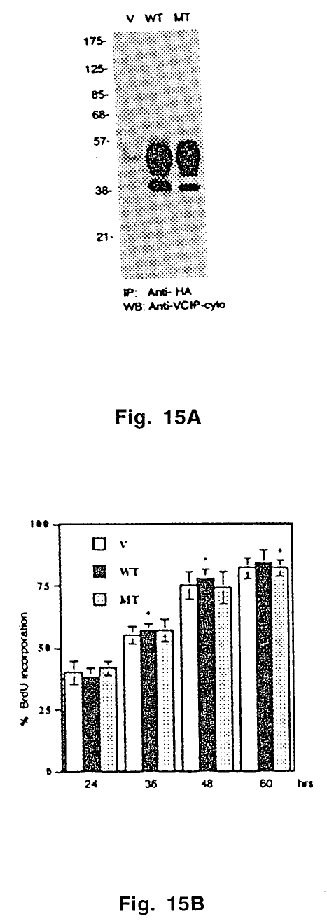

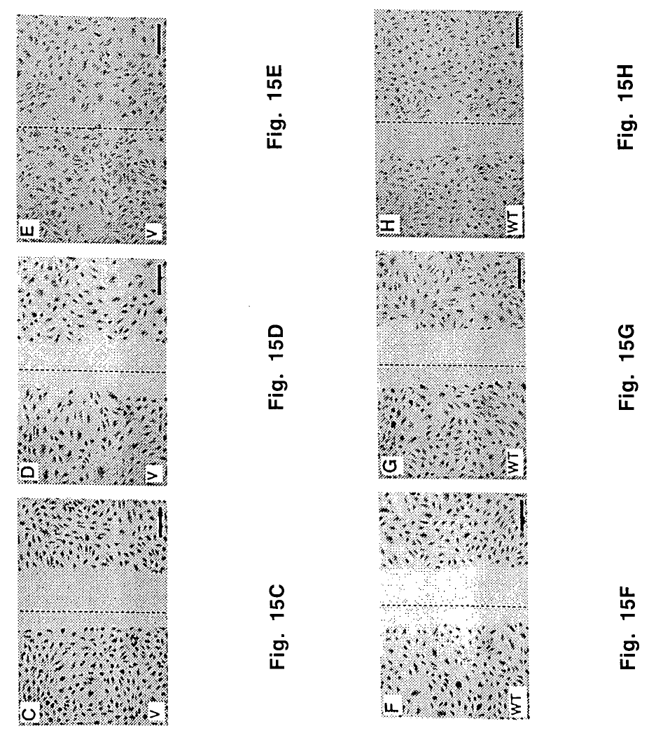

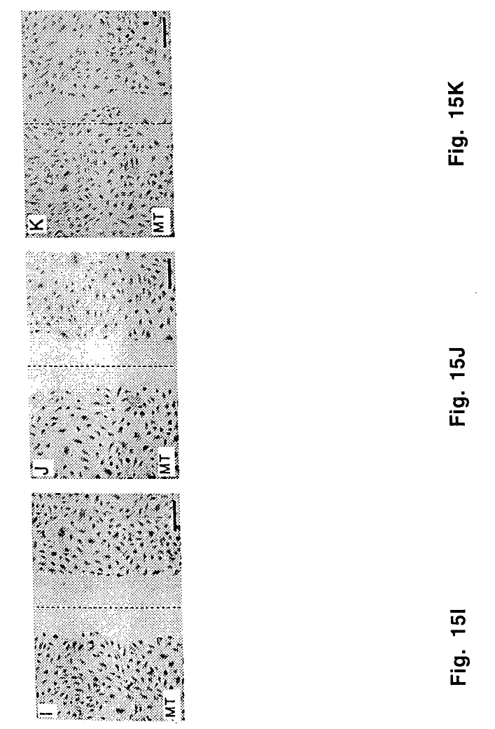

HUVECs from passage 3-4 were used for the expression of control (V), wild-type PAP2b-RGD (WT), or mutant PAP2b-RGE (MT) by viral transduction. MT-PAP2b-RGE represents a single mutation at position D 184E. To monitor the non-toxicity of the viral particles used for infection, control supernatants generated from vector alone (construct A) were used. Expression levels were determined by immunoprecipitation and western immunoblot assay showing comparable levels of wild-type (construct B) and mutant (construct C) proteins (FIG. 15A). As determined by Brdu incorporation and monitored for the indicated time period, there was no defects observed in cellular growth between the control, MT or WT expressing cells (FIG. 15B).

To determine the effects of VCIP on endothelial cells migration, wounded cells were incubated in a defined media and their ability to repopulate the wounded area was monitored for 0, 5, and 10 hours. About 40-60% confluent HUVECs on 12-well culture plates were infected with vector alone, PAP2b-WT or PAP2b-MT retroviral particles overnight. Next day the cells were replenished with fresh media and allowed to grow to form confluent monolayer for about 12-24 hrs. Confluent monolayer cells were injured by sterile 200 μl micropipet tip, washed twice in sterile PBS, and allowed to recover in defined M 199 media. After specific times the plates were removed, washed in PBS and fixed in 4% paraformaldehyde. Fixed cells were stained with eosin/hematoxylin and images were documented using Zeiss phase contrast microscope.

As shown in FIG. 15, vector alone control (construct A) cells were able to recuperate and migrate through injured region by the 10th hour (FIGS. 15C, E). Cells expressing PAP2b-wild-type (construct B) migrated at a reduced speed and showed delayed wound closure (FIGS. 15F-H). Conversely, cells expressing mutant PAP2b (construct C) appeared to have no defects in cell migration (FIGS. 15I-K); by 18-24 hrs they appear to close up the wound.

Furthermore, Transwell cell migration assay was performed using VCIP infected cells. Migration assay was carried out using modified chemotactic Transwell Boyden (8.0 μM) chambers (Schor et al., 1996). Endothelial cells infected with retroviral constructs were detached non-enzymatically, washed once with complete media, followed with PBS, and resuspended in defined M199 media (M199+1×ITS [insulin, transferrin and selenium-A]. Top chamber was filled with 500 μl media containing 2.5×10⁴ cells and the lower chamber was filled with 500 μl of defined media. Following 6 hours at 37° C. in CO₂ incubator, cells that remained on the upper chamber were gently removed by cotton Q-tips. Cells that migrated to the lower side of filter were fixed with 4% paraformaldehyde and stained with 0.5% crystal violet. Cell number was counted using a phase contrast microscope. A minimum of 10 random fields at 100× magnification were selected for each chamber-filter. Experiments were performed three times with triplicates.

There was no reduction in the rate of migration of cells expressing PAP2b-wild-type as compared to control and mutant cells (<1.0 fold). Wound healing assay requires both solitary as well as collective cell movement. In contrast, Transwell Boyden chamber measures solitary cell movement. Thus, it appears that PAP2b/VCIP may impede collective but not single cell movement.

### EXAMPLE 22

Interaction of PAP2b/VCIP With p120catenin

This example examines the biochemical basis for the effects of PAP2b/VCIP on endothelial cell migration. Molecular organization of cell-cell contacts often requires participation of junction proteins and cytoskeletal elements. Besides the extracellular regions harboring phosphatase activity domain and the putative integrin-binding RGD motif, sequence analysis of the cytoplasmic domains of PAP2b/VCIP revealed no known protein binding motifs or enzymatic features. Therefore, PAP2b/VCIP interaction with known intracellular proteins that may be involved in cell-adherent junctional organization was examined. To do this, monolayer HUVECs infected with full length PAP2b (WT) and stimulated with VEGF¹⁶⁵ were solubilized in modified RIPA buffer, clarified, and subjected to coimmunoprecipitation analysis.

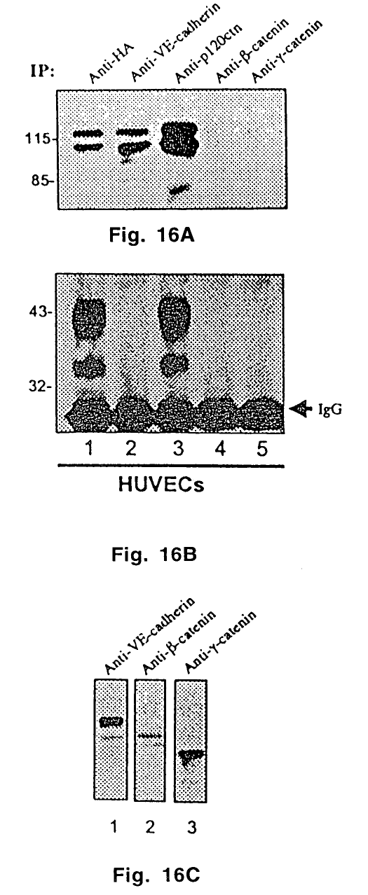

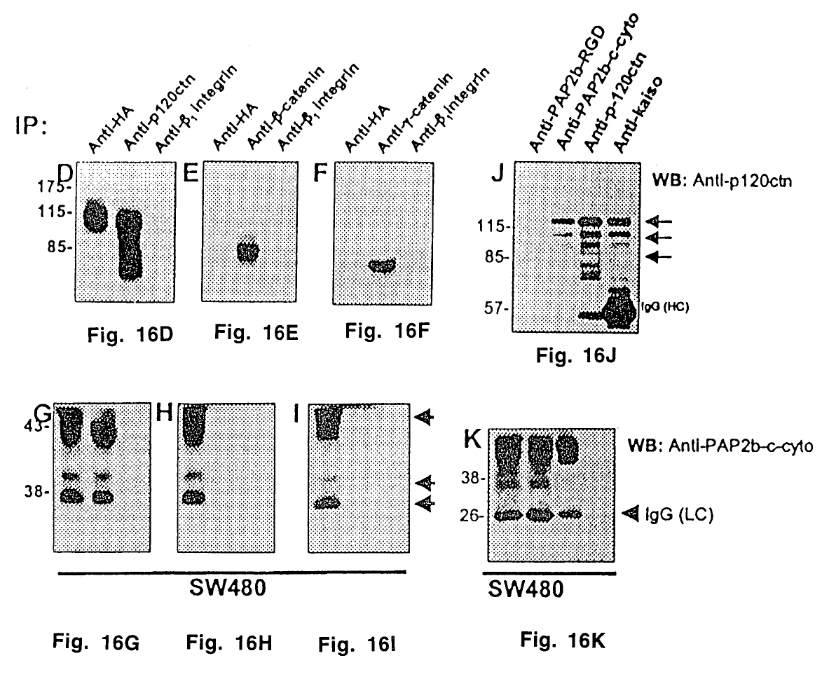

Interestingly, anti-p120catenin coprecipitated VCIP and vice versa (FIG. 16A, lanes 1 and 3). In contrast, anti-VE cadherin, anti-β-catenin, and anti-γ-catenin did not show VCIP immunoreactivity even after prolonged exposure (FIG. 16A, lanes 2, 4 and 5). p120catenin has been reported to associate with P3- and γ-catenins, however, only trace amount of p120catenin was detected on prolonged exposures, which may be due to the stringent washing buffer employed in the assays. Individual lanes (lanes 2, 4 and 5 from FIG. 16A) were excised and reprobed with anti-VE-cadherin, anti-β-catenin and anti-γ-catenin monoclonal antibodies to determine the presence of respective polypeptide chains (FIG. 16C). In parallel experiment, western blotting analysis of identical blots with anti-VEGF receptor-2 (VEGFR-2), anti-Tie-2, anti-CD36, and PECAM-1 antibodies failed to detect any one of these cell surface proteins.

### EXAMPLE 23

E-Cadherin Independent Interaction of PAP2b/VCIP With p120catenin

p120catenin, which was initially identified as a V-Src substrate, has been implicated in cell-cell adhesion, signaling and tumor progression. Because p120catenin is known to interact with cadherin when present in the cell-cell junction, the association of PAP2b/VCIP with p120catenin was examined. For this purpose, E-cadherin deficient SW480 (human colon carcinoma) cells were made to stably express WT-PAP2b prior to p120catenin interactions studies. Cell lysates prepared in RIPA buffer were clarified and immunoprecipitated with anti-HA, anti-p120ctn, anti-β-catenin, anti-γ-catenin and anti-β1 monoclonal antibodies.

The anti-HA and anti-p120ctn monoclonal antibodies co-precipitated p120ctn and PAP2b/VCIP reciprocally (FIG. 16D). Similar to results using primary endothelial cells, anti-PAP2b immunoblots did not contain anti-β-catenin or anti-γ-catenin immunoreactivities (FIGS. 16E and F). Anti-β1 integrin antibody was included as a negative control for immunoprecipitation. Comparable expressions of PAP2b proteins were evaluated by immunoblotting with anti-PAP2b-c-cyto polyclonal antibody (FIGS. 16G, H and I).

To further analyze the specific interaction of PAP2b/VCIP with p120catenin, lysates prepared from SW480 cells expressing PAP2b-wild-type (construct B) were immunodepleted with anti-pan-cadherin mAb. Cadherin depleted samples were subjected to immunoprecipitation with indicated antibodies and analyzed as shown in FIGS. 16J and K. Together, these data suggest that the interaction of PAP2b with p120ctn is specific.

### EXAMPLE 24

The Cytoplasmic Domain of PAP2b/VCIP Interacts Directly With p120catenin

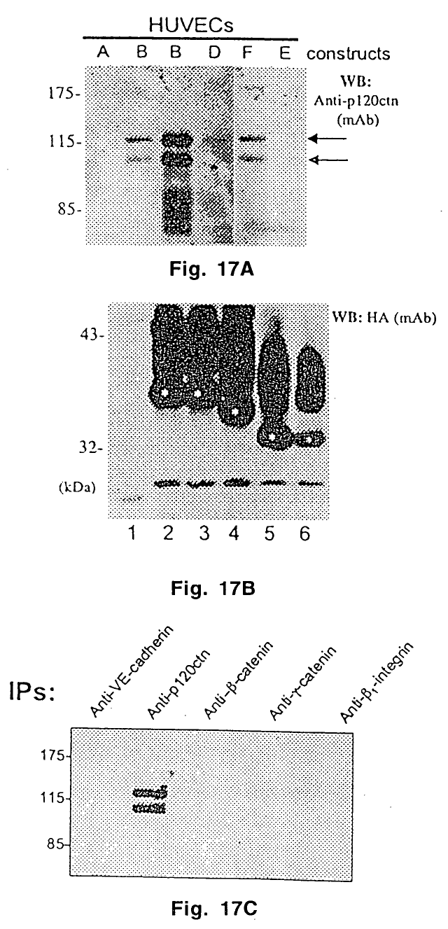

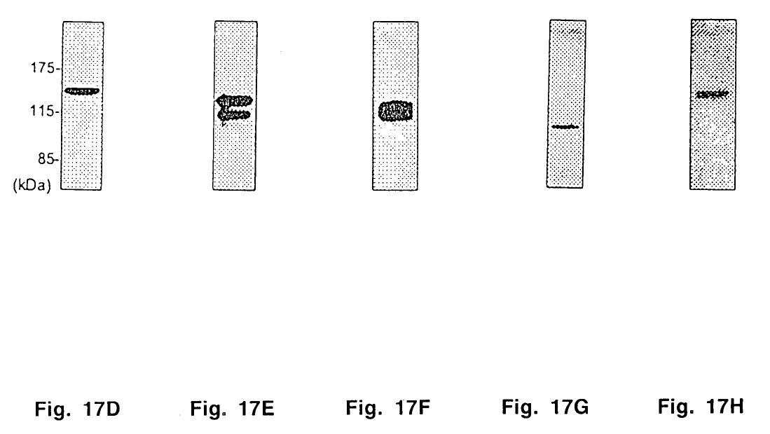

This example determines the region of VCIP/PAP2b that specifically interacts with p120catenin. To examine this question cell lysates prepared from endothelial cells infected with various retroviral PAP2b constructs were subjected to coimmunoprecipitation analysis (FIGS. 17A-B). Cell extracts were immunoprecipitated with anti-HA (lanes 1, 2, 4, 5 and 6) or anti-p120 catenin (lane 3) monoclonal antibodies (mAb) and subjected to western immunoblot analysis with anti-p120 catenin (FIG. 17A). To determine the level of expression, the bottom portion of the blot was analyzed by immunoblotting with anti-HA mAbs (FIG. 17B).

Anti-HA monoclonal antibody did not co-precipitate any detectable PAP2b or p120ctn from cells infected with vector alone (construct A) (FIGS. 17A-B, lane 1). Anti-HA mAb coprecipitated p120ctn immunoreactivities from cells infected with constructs B, D, F but not E (constructs are shown in FIG. 14). Construct E lacks entire C-terminal cytoplasmic segment of VCIP.

To further substantiate that the C-terminal cytoplasmic domain of PAP2b was indeed the region involved in the interaction, far-western assay (Wary et al., 1996) was performed using recombinant affinity purified GST-PAP2b-cyto fusion protein as follows. Briefly, two 15-cm dish of HUVECs, i.e., about 1.0-1.2 mg protein per data point, were used. Cells were solublized in modified RIPA buffer (50 mM HEPES, pH 7.5, 1% Triton X-100, 0.1% SDS, 0.25% sodium deoxycholate, 150 mM sodium chloride, 5 mM magnesium chloride, 1 mM calcium chloride, and 14 g/ml leupeptin, 1 μg/ml pepstatin, and 1 mM phenylmethylsulfonyl fluoride). Immunopecipitation was performed as described above. After blocking with 5% milk in TBS (25 mM Tris, 150 mM NaCl, pH 7.5), the membrane was incubated with soluble Gst-VCIP/PAP2b-cyto fusion protein (2 μg/ml in TBS, Tween 0.1%) in the presence of 2 mM DTT. After rinsing with TBS, pH 7.5, the membrane was incubated with anti-Gst mouse monoclonal antibodies and detected as previously described (Schor et al., 1996). For immunoprecipitation, antibodies were used at 3-5 μg per data point. For western analysis, antibodies were prepared in 3% BSA, in TBS, pH 7.5 at a concentration of 0.5 μg/ml and 2.0 μg/ml for mouse monoclonal and rabbit polyclonal antibodies, respectively.

As shown in FIG. 17C, the results clearly indicated that the C-terminal cytoplasmic region of PAP2b/VCIP was sufficient to interact with p120catenin. This association is considered specific since the GST-PAP2b-c-cyto fusion protein did not react with VE cadherin, β- and γ-catenin, or β1-integrin polypeptides (FIG. 17C). This blot was stripped, and individual lanes were excised and analyzed for the presence of VE-cadherin, p120ctn, β-catenin, γ-catenin, and β1-integrin polypeptides (FIGS. 17D, E, F, G, H), respectively.

### EXAMPLE 25

Expression of PAP2b/VCIP Regulates p120 Catenin and β-Catenin

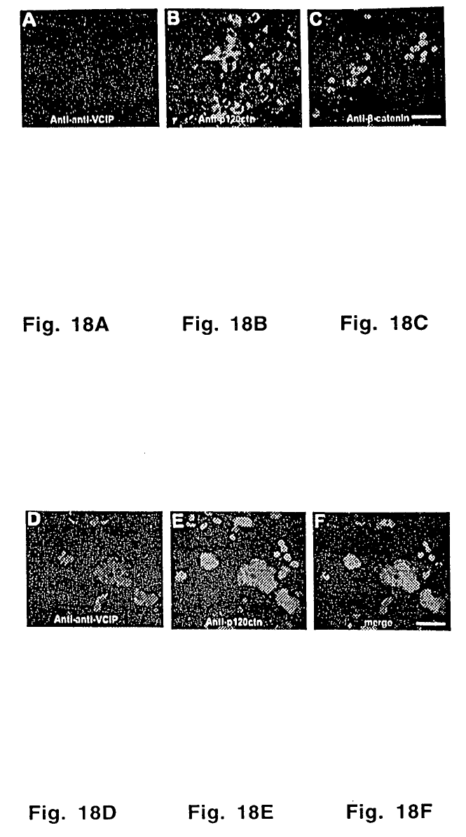

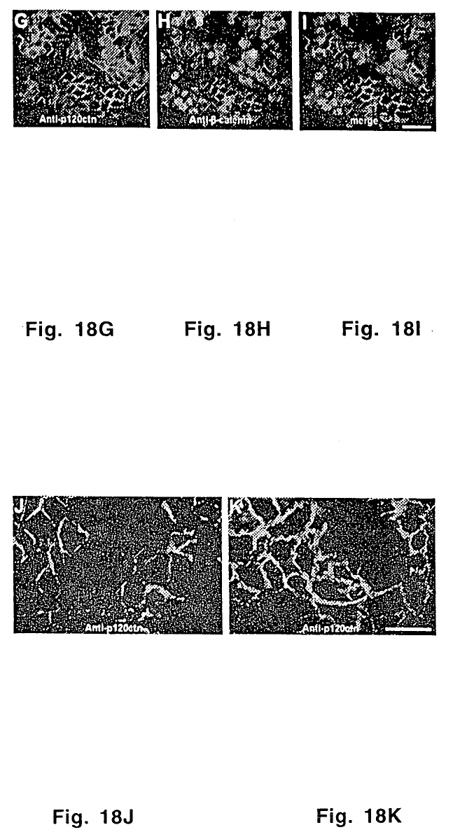

SW480 cells stably expressing various PAP2b/VCIP constructs (constructs are shown in FIG. 14) were used. To measure the capacity of PAP2b-WT to bind p120catenin, its ability to recruit p120catenin at the pericellular cavity was evaluated. Endogenous PAP2b protein was undetectable in SW480 cells (FIG. 18A). In parental SW480 cells, endogenous p120catenin remained evenly distributed in the cytoplasm (FIG. 18B), whereas β-catenin was exclusively localized in the nucleus (FIG. 18C). Expression of wild-type PAP2b resulted in recruitment of p120catenin to the cell-cell contact sites (FIGS. 18D-F). Interestingly, this event was also accompanied by an increased translocation of β-catenin to the cell-cell contact sites (FIGS. 18G-I).

Next, to determine whether this event also occurs in HUVECs, endothelial cells were infected with construct A (vector alone) or construct B (Wild-type PAP2b) for 12 hours and subjected to immunofluorescence labeling with anti-p120catenin antibody. The results showed that expression of PAP2b in HUVECs significantly increased recruitment of p120catenin into the pericellular cavity of the plasma-membrane (FIGS. 18J, K). By indirect epifluorescence microscopy, no alteration of β-catenin redistribution was observed in HUVECs overexpressing PAP2b constructs (data not shown). Thus, if in fact p120catenin modulates the activities of small GTPases including RhoA, Rac, and Cdc42 as previously described, then the present data suggest a mechanism for regulating the balance between the adhesive and motile phenotypes.

### EXAMPLE 26

PAP2b/VCIP Modulate LEF-1 Transcriptional Activities in SW480 Cells

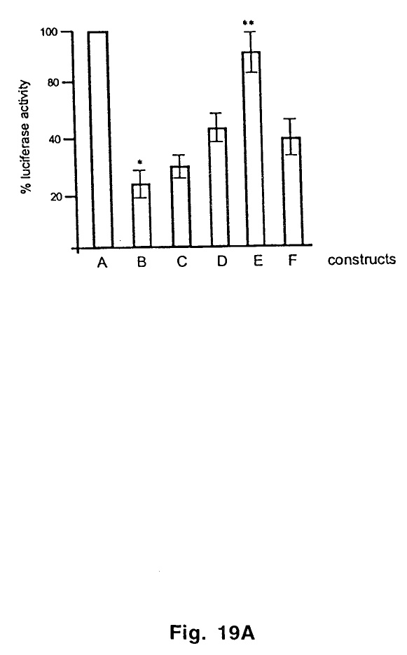

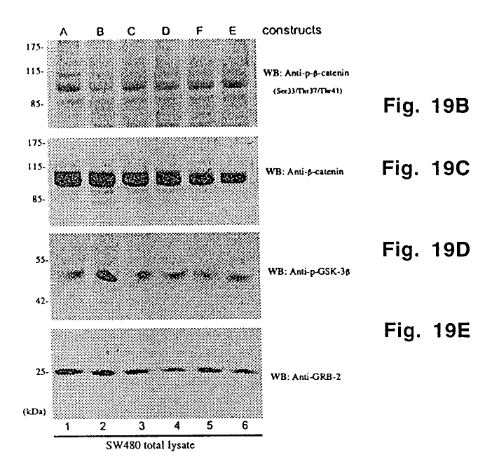

Recent study suggests that activity of TCF is negatively regulated by the actions of PAP2b/VCIP. Increased phosphorylation states of GSKβ (ser-9) and β-catenin proteins suggested a link between PAP2b/VCIP and TCF nuclear function. Beta-catenin is a co-activator of LEF-1 in stimulating transcription from multimerized LEF-1 binding sites. To investigate the possibility that PAP2b and p120catenin interact to modulate β-catenin-dependent LEF-1 activity, a LEF-1 reporter assay employing luciferase reporter constructs containing multimeric LEF-1 binding sites (TOPFLASH) was used. LEF-1 reporter assay has been described previously (Xia et al., 2001). At least three independent clones of SW480 cells were employed for the LEF-1 assay. As shown in FIG. 19A, cotransfection of TOPFLASH with LEF-1 resulted in increased luciferase activity, which was adjusted to 100% (control). Cotransfection of TOPFLASH into cells expressing constructs B, C, D, F and H (FIG. 14) reduced luciferase activities by ˜3.2, 4.2, 4.9, 1, and 1.2 folds. In contrast, construct G did not inhibit LEF-1 luciferase activity significantly.

The phosphorylation states of β-catenin were analyzed in cell lysates by immunoblotting with anti-phosphospecific antibodies against the Ser-33 and Thr-37/47 amino acid residues (FIG. 19B). The basal phosphorylation state of β-catenin appeared to be significant in control cells FIG. 19B (lane 1). In contrast, over-expression of wild-type PAP2b in SW480 cells significantly reduced phosphorylation of β-catenin (FIG. 19B, lane 2). SW480 cells expressing construct-C, D, F and E (lane 3, 4, 5 and 6) showed moderate increase in the phosphorylation state of β-catenin, but not significantly higher than basal level (lane 1). In addition, over-exression of wild-type PAP2b (construct B) induced increased phosphorylation of GSK30 (Ser9) kinase protein at least by ˜2.5 fold (FIG. 19D, lane 2). In contrast, there was no significant alteration in the phosphorylation state of GSK3β in lanes 1, 3, 4, 5 and 6 (FIG. 19D). Equal loading of proteins were determined by immunoblotting with anti-GRB-2 antibody (FIG. 19E). These data suggest that the functions of β-catenin may be regulated by PAP2b/p120catenin molecular interactions, which could modulate LEF-1 transcriptional activities.

### EXAMPLE 27

VCIP Potentiates Tumor Growth by Promoting Tumor Angiogenesis

It is known that the growth of tumors beyond 2-3 mm size requires formation of their own individual blood supply. Without oxygen and nutrients most tumor will remain dormant for a prolonged period of time. However, upon activation of an “angiogenic switch” such dormant tumors will become vascularized and grow, and eventually form metastatic foci at distant site. To demonstrate the role of VCIP in tumor growth and angiogenesis, xenograft experiments in immunocompromized athymic nude mouse model was performed as described below.

Human colon carcinoma cells (SW480) expressing HA-tagged vector alone (control), wild-type VCIP (RGD), mutant VCIP (RGE), mutant VCIP-phosphatase dead and mutant VCIP-cyto constructs were maintained in DMEM containing 7.5% FBS and 200 μg ml⁻¹ geneticin (G418). Generation of constructs, infections and establishment of stable lines have been described above. The expression of PAP2b/VCIP protein was confirmed by Western blotting prior to injection to the mice.

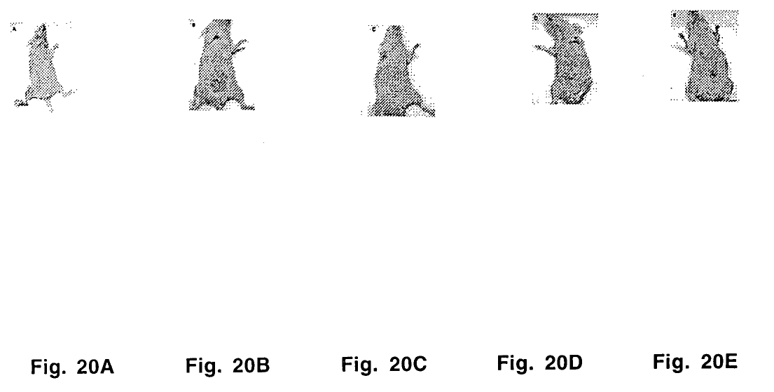

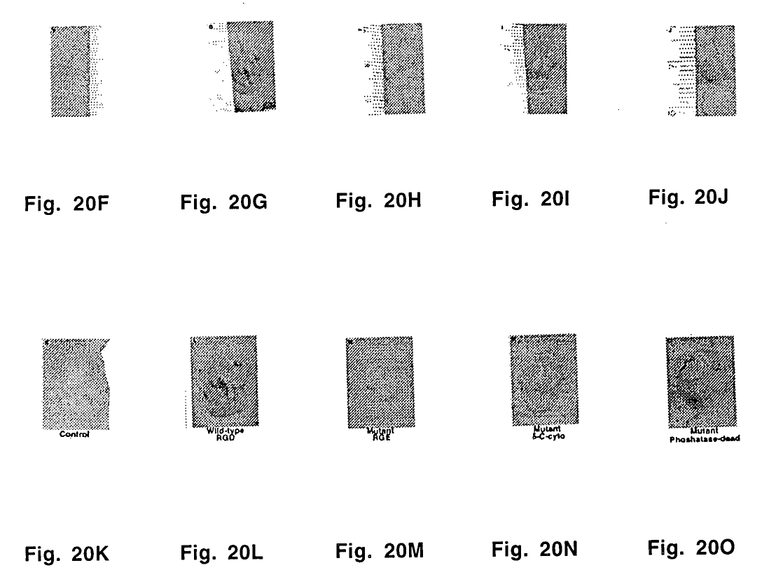

As shown in FIG. 20, SW480 control cells (vector alone) did not grow significantly (diameter=0.2 cm±0.08, n=4). In contrast, SW480 cells expressing VCIP-RGD (WT) grew drammatically within 30 days with visibly distinct vasculature (angiogenic blood vessels) and localized hemorrhage, suggesting aggressive nature of tumor growth. The sizes of wild-type VCIP tumors were of diameter 2.1±0.5 cm (n=3). SW480 cells expressing VCIP-RGE mutants showed reduced tumor growth with no visible vasculature (lack of angiogenesis). The tumor sizes were of 4.2+0.8 cm, n=3. In contrast, SW480 cells expressing VCIP-Δ-C-cyto, a mutant that lacks the C-terminal cytoplasmic portion, showed robust tumor growth and neovasculature formation comparable to VCIP-RGD-WT. Interestingly, SW480 cells expressing phosphatase-inactive (phosphatase-dead) form of VCIP induced tumor growth and extensive angiogenesis. A ruler in centimeter scale was placed next to the tumor mass to show the extent of tumor growth (FIGS. 20 F, G, H, I and J). FIGS. 20 K, L, M, N and 0 showed close-up images of the experimental tumor in nude mice.

### EXAMPLE 28

VCIP Augments Tumor Metastasis

The impact and extent of tumor growth and tumor angiogenesis can also be determined by metastatic foci formation at distant sites. To study minimal metastasis spread at 30 days, brain, lung, liver, spleen, and kidney tissues were subjected to PCR to identify the presence of human tumor cells as determined by human Alu sequences. One of the main advantages of this assay is its sensitivity, i.e. as few as 50 tumor cells per 108 host cells can be detected.

Tissues from sacrificed mice were snap-frozen in liquid-nitrogen and stored at −80° C. for later use. DNA was purified using Qiagen RNA/DNA mini kit according to manufacturer's instruction (Qiagen, Inc.). Purified DNAs were dissolved in deionized water, quantified and stored at −20° C. until use.

In order to detect metastatic human SW480 cells expressing various VCIP contructs in the mouse tissues, ALU-PCR strategy was employed. The oligonucleotides for human ALU were: sense, 5′-GTTGCCCAAGTTGGAGTGCAATGG-3′ (SEQ ID NO:33) and antisense, 5′-ACAATGGCTCACGCCTGTAATCCC-3′ (SEQ ID NO:34). Ten nanograms each of genomic DNA extracted from various mouse tissues were used in a final 25 μl reaction using Taq PCR master mix Kit (Qiagen, Inc). The PCR parameters were set as initial denaturation 94° C., 10 min; denaturation 94° C., 1.5 min; annealing 55° C., 1.5 min; and extension 72° C., 2 min for 25 cycles followed by final extension of 72° C. for 7 min. For internal control, mouse glyceralaldehyde-3-phosphate dehydrogenase (GAPDH) was included using primers: sense, 5′-TGGAGTCTACTGGTGTCTTCACCACCATG-3′ (SEQ ID NO:35) and antisense, 5′-GCAGGAGACAACCTGGTCCTCAGTG-3′ (SEQ ID NO:36).

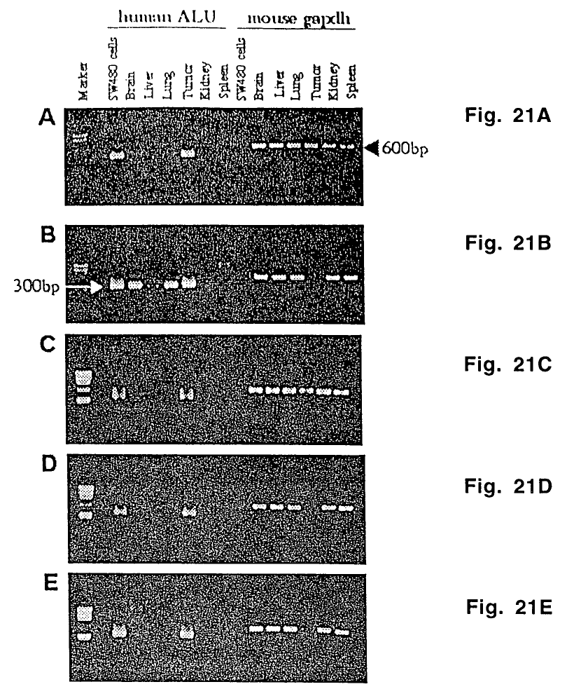

As shown in FIG. 21, brain, liver, lung, kidney and spleen tissues obtained from control mice did not amplify human ALU 300 bp PCR product. Positive ALU DNA PCR product was found in SW480 control cells and primary tumors. In contrast, brain, liver, and lung mouse tissues obtained from mice injected with SW480 cells expressing wild-type VCIP/PAP2b-RGD construct produced positive 300 bp ALU PCR product, but not in kidney and spleen (FIG. 21B). Although there were tumor growth in other set of mice that were administered with mutant constructs, no metastatic SW480 cell was detected in any of the tissues recovered as illustrated by lack of human ALU PCR product (FIGS. 21C, D, and E). Murine GAPDH PCR 600 bp product served as a control for tissues and PCR conditions employed. These data indicte that wild-type VCIP-RGD supports tumor metastasis, whereas various VCIP mutants do not.

### EXAMPLE 29

Anti-VCIP Antibody Blocks Angiogenesis in vitro

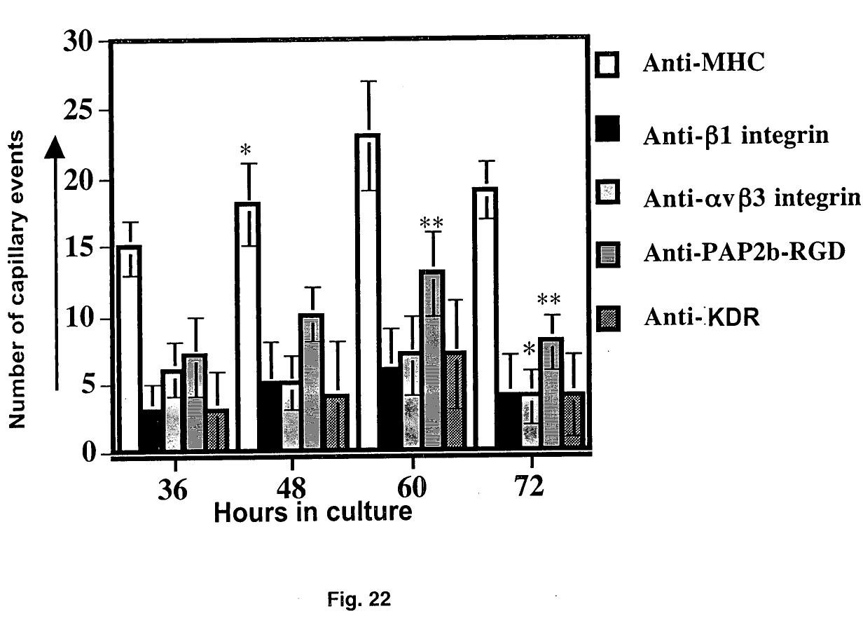

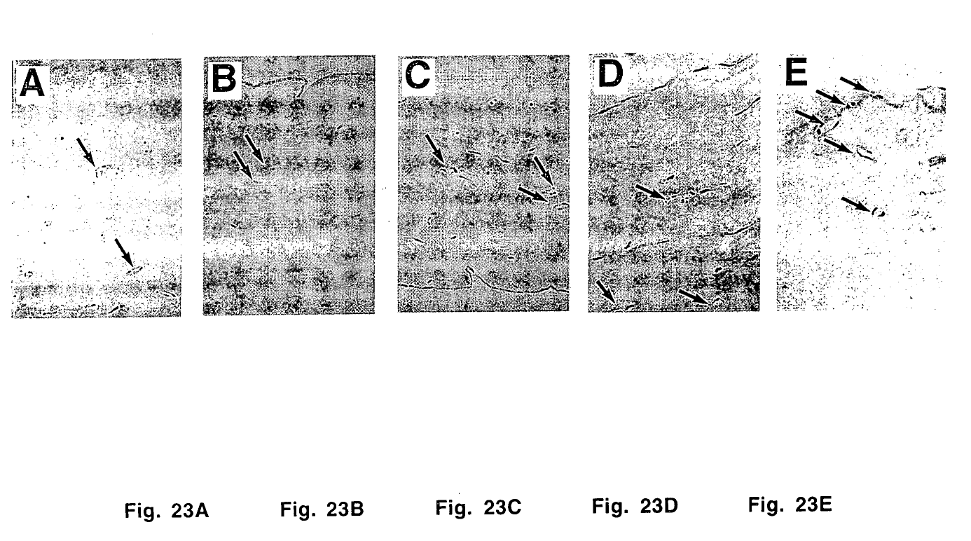

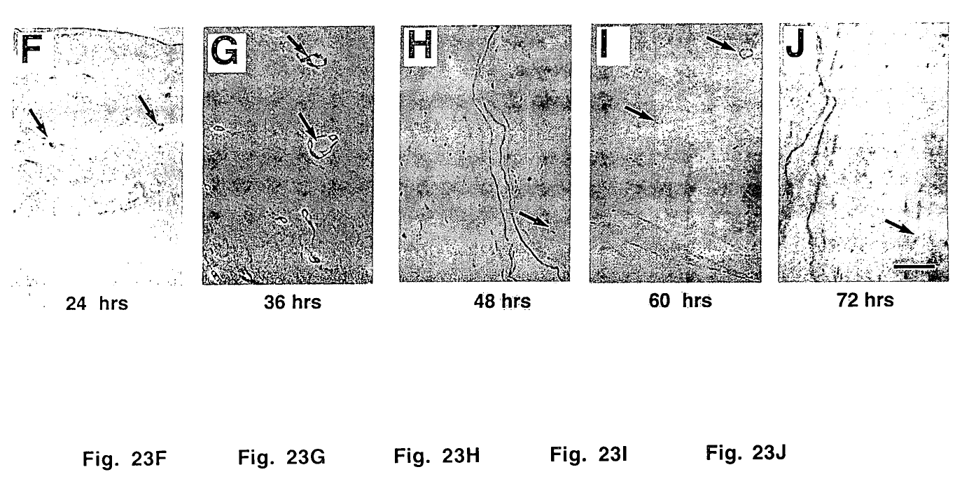

Capillary morphogenesis of endothelial cells was performed in a 3D type I collagen matrix as described previously (Humtsoe et al., 2003) to evaluate the ability of anti-PAP2b-RGD (anti-VCIP-RGD) and PAP2b/VCIP derived peptides to inhibit morphogenic differentiation of endothelial cells. The vessels formed at the end of 24 hours of culture in 3D collagen were considered “pre-formed vessels”, while capillaries formed after 24 hours of culture were considered as “new capillaries”. The starting number of pre-formed capillaries at 24 hours was 34.8±4.5 (n=8). In this assay, specific monoclonal antibodies were added to endothelial cells already cultured for 24 hours in 3D type I collagen matrices with VEGF¹⁶⁵. Anti-MHC class II (W6/32) and anti-αᵥβ₃ monoclonal antibodies (LM609) were used as negative and positive controls, respectively. Whereas anti-αᵥβ₃ monoclonal antibodies reduced the number of interconnections and induced the regression of preformed capillaries (FIG. 22 & FIG. 23F-J), anti-MHC class II monoclonal antibodies had minimal effect (FIG. 22 & FIG. 23A-E). Assays were also performed without antibodies as well as with mouse immunoglobulins as controls to illustrate that anti-MHC class II monoclonal antibodies had minimal effect in the assay (data not shown).

Anti-α_{c}β₃ monoclonal antibodies only reduced the number of pre-formed capillaries by less than 50%, suggesting that other cell surface proteins, such as the fibronectin-binding integrin α₅β₁ and collagen/laminin binding integrins might also play a role in capillary morphogenesis. Indeed, anti-β_integrin subunit monoclonal antibodies inhibited pre-formed interconnections and reduced the number of capillaries by 60-70% (FIG. 22). Furthermore, anti-α₂β₁ integrin monoclonal antibodies reduced the number of capillaries by 45% to 60% over the period of 36 to 72 hours, suggesting that the collagen-binding α₂β₁ integrin was the major receptor involved in signaling from the type I collagen matrix used in this assay (data not shown). It remains possible that type I collagen receptors α₁₀β₁ and α₁₀β₁ integrins could also play a role. No two monoclonal antibodies were added together, since it has been reported that α₅β₁ and αᵥβ₃ integrin antibodies together induce complete collapse and regression of tubules in vitro.

Anti-PAP2b-RGD (anti-VCIP-RGD) monoclonal antibodies inhibited the formation of new capillaries, reducing the number of capillaries by ˜45% to 55% after 60 to 72 hours. Anti-KDR and anti-VE-cadherin monoclonal antibodies reduced the number of capillaries by 45% to 75% over the period of 36 to 72 hours. It is likely that anti-KDR and anti-VE-cadherin monoclonal antibodies blocked different signaling pathways. Whereas VEGF-induced KDR signaling is required for cell proliferation, survival, and differentiation, as well as vascular permeability, VE-Cadherin is required for the maintenance of cell-cell junctions and for cell polarization. Antibodies that affect any one aspect of the endothelial cell activation and differentiation pathway are likely to inhibit capillary morphogenesis in vitro.

The following references were cited herein:
Gottardi et al., Biotinylation and assessment of membrane polarity: caveats and methodological concerns. Am. J. Physiol. 268:F285-F295 (1995).
Humtsoe et al., Regulation of cell-cell interactions by phosphatidic acid phosphatase 2b/VCIP. EMBO J. 22:1539-1554 (2003).
Kai et al., Cloning and characterization of two human isozymes of Mg²⁺-independent phosphatidic acid phosphatase. J. Biol. Chem. 272:24572-24578 (1997).
Mainiero et al., Signal transduction by the (α6β4 integrin: distinct β4 subunit sites mediate recruitment of Shc/Grb2 and association with the cytoskeleton of hemidesmosomes. EMBO J. 14:4470-4481 (1995).
Mainiero et al., The coupling of α6β4 integrin to Ras-MAP kinase pathways mediated by Shc controls keratinocyte proliferation. EMBO J. 16:2365-2375 (1997).
Niessen and Gumbiner, Cadherin-mediated cell sorting not determined by binding or adhesion specificity. J. Cell Biol. 156:389-399 (2002).
Orlando and Cheresh, Arginine-glycine-aspartic acid binding leading to molecular stabilization between integrin αvβ3 and its ligand. J. Biol. Chem. 266:19543-1955 (1991).
Ron and Dressler, pGSTag—a versatile bacterial expression plasmid for enzymatic labeling of recombinant proteins. Biotechniques 13:866-869 (1992).
Schor et al., Substratum-dependent stimulation of fibroblast migration by the gelatin-binding domain of fibronectin. J Cell Sci. 109:2581-2590 (1996).
Wary et al., A homozygous deletion within the carbonic anhydrase-like domain of the Ptprg gene in murine L-cells. Cancer Res. 53:1498-1502 (1993).
Wary et al., The adaptor protein Shc couples a class of integrins to the control of cell cycle progression. Cell 87:733-743 (1996).
Wary et al., A requirement for caveolin-1 and associated kinase Fyn in integrin signaling and anchorage-dependent cell growth. Cell 94:625-634 (1998).
Wary et al., Biochemical analysis of integrin-mediated Shc signaling. In Methods in Molecular Biology, Howlett ed., Humana Press, Totowa, N.J., 35-49 (1999a).
Wary et al., Specificity of integrin signaling. In Signaling through Cell Adhesion Molecules, Guan ed., CRC Press, Boca Raton, Fla., 101-114 (1999b).
Xia et al., Regulation of the p21-activated kinase (PAK) by a human Gbeta-like WD-repeat protein, hPIP1. Proc. Natl. Acad. Sci. USA. 98:6174-6179 (2001).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A method of enhancing cell-cell interaction, comprising the step of expressing in a cell a vascular endothelial growth factor and type I collagen inducible protein (VCIP) having the sequence of SEQ ID NO: 13.

2. The method of claim 1, wherein said cell-cell interaction is mediated by integrin ligand.

3. The method of claim 1, wherein said cell has altered motility as a result of expressing said VCIP.

4. The method of claim 1, wherein said VCIP is encoded by a gene delivered to said cell by a viral vector or a non-viral gene delivery system.

5. The method of claim 4, wherein said viral vector is an adenoviral vector or a retroviral vector.

6. The method of claim 4, wherein said non-viral gene delivery system is liposome or a high pressure gene delivery system.

7. The method of claim 1, wherein said cell-cell interaction contributes to a biological process selected from the group consisting of normal cell cycle progression, unwanted cell cycle progression, vascular malformation, expansion of atherosclerotic lesions, invasion of tumor cells, inflammation, cell motility, and angiogenesis.

8. A method of inhibiting cell-cell interaction, comprising the step of blocking the binding of integrins to cell surface vascular endothelial growth factor and type I collagen inducible protein (VCIP).

9. The method of claim 8, wherein said cell-cell interaction is mediated by integrin ligand.

10. The method of claim 8, wherein said binding is blocked by an antibody directed against a peptide derived from said VCIP, wherein said peptide comprises a CRGDD sequence.

11. The method of claim 10, wherein said peptide has the sequence of SEQ ID NO:2.

12. The method of claim 8, wherein said binding is blocked by a peptide derived from said VCIP, wherein said peptide comprises a CRGDD sequence.

13. The method of claim 12, wherein said peptide has a sequence of SEQ ID NO:20 or SEQ ID NO:23.

14. The method of claim 8, wherein said cell-cell interaction contributes to a biological process selected from the group consisting of normal cell cycle progression, unwanted cell cycle progression, vascular malformation, expansion of atherosclerotic lesions, invasion of tumor cells, inflammation, cell motility, and angiogenesis.

15. A method of treating a patient having a pathological condition resulted from integrin-mediated cell-cell interaction, said method comprises the step of administering to said patient an agent that blocks the binding of integrin to cell surface vascular endothelial growth factor and type I collagen inducible protein (VCIP).

16. The method of claim 15, wherein said binding is blocked by an antibody directed against a peptide derived from said VCIP, wherein said peptide comprises a CRGDD sequence.

17. The method of claim 16, wherein said peptide has the sequence of SEQ ID NO:2.

18. The method of claim 15, wherein said binding is blocked by a peptide derived from said VCIP, wherein said peptide comprises a CRGDD sequence.

19. The method of claim 18, wherein said peptide has the sequence of SEQ ID NO:20 or SEQ ID NO:23.

20. The method of claim 15, wherein said cell-cell interaction contributes to a biological process selected from the group consisting of normal cell cycle progression, unwanted cell cycle progression, vascular malformation, expansion of atherosclerotic lesions, invasion of tumor cells, inflammation, cell motility, and angiogenesis.

21. The method of claim 15, wherein said pathological condition is selected from the group consisting of stroke, thrombosis, tumor growth, metastasis, arthritis, cardiac infarction, psoriasis, diabetic retinopathy, inflammation, and angiogenesis.

22. A peptide derived from vascular endothelial growth factor and type I collagen inducible protein (VCIP) that has the sequence of SEQ ID NO:13, wherein said peptide comprises a CRGDD sequence.

23. The peptide of claim 22, wherein said peptide has 49 amino acid residues.

24. The peptide of claim 22, wherein said peptide has the sequence of SEQ ID NO: 20 or 32.

25. A vector comprising nucleotide sequence that encodes the peptide of claim 22.

26. The vector of claim 25, wherein said peptide has the sequence of SEQ ID NO: 20 or 32.

27. An antibody directed against the peptide of claim 22.

28. The antibody of claim 27, wherein said peptide has the sequence of SEQ ID NO: 2.

29. A diagnostic kit useful for detecting vascular endothelial growth factor and type I collagen inducible protein (VCIP) in an individual, said kit comprises an antibody directed against said VCIP or against a fragment of said VCIP.

30. The kit of claim 29, wherein said fragment of VCIP has a sequence selected from the group consisting of SEQ ID NOs: 2, 20 and 32.

31. The kit of claim 29, wherein said individual has a disease selected from the group consisting of pathological angiogenesis, inflammation, arthritis, psoriasis, atherosclerosis, and metastatic disease.

32. A method of inhibiting angiogenesis and the formation of capillaries in a patient in need of such treatment, comprising the step of administering to said patient a pharmacological effective amount of antibody directed against a peptide derived from vascular endothelial growth factor and type I collagen inducible protein, wherein said peptide comprises a RGD sequence.

33. The method of claim 32, wherein said peptide has the sequence of SEQ ID NO: 2.

34. A method of inhibiting angiogenesis and the formation of capillaries in a patient in need of such treatment, comprising the step of administering to said patient a pharmacological effective amount of a peptide derived from vascular endothelial growth factor and type I collagen inducible protein, wherein said peptide comprises a RGD sequence.

35. The method of claim 34, wherein said peptide has the sequence of SEQ ID NO:20 or SEQ ID NO:23.

36. A method of inhibiting angiogenesis and the formation of capillaries in a patient in need of such treatment, comprising the step of administering to said patient a pharmacological effective amount of antisense oligonucleotides against the transcripts encoding vascular endothelial growth factor and type I collagen inducible protein.

37. A method of enhancing cell-cell adhesion junction formation in a patient, comprising the step of administering to said patient a vector encoding vascular endothelial growth factor and type I collagen inducible protein (VCIP).

38. The method of claim 37, wherein said vector is a viral vector or a non-viral gene delivery system.

39. The method of claim 37, wherein said patient has compromised blood-brain barrier functions.

40. A method of enhancing angiogenesis in a patient, comprising the step of administering to said patient a vector encoding vascular endothelial growth factor and type I collagen inducible protein (VCIP).

41. The method of claim 40, wherein said vector is a viral vector or a non-viral gene delivery system.


SEQUENCE LISTING (36 sequences)

SEQ ID NO: 1 - protein, 15 aa, Unknown
  CHAIN: peptide used to raise anti-VCIP-cyto-C16 antibody
LSPVDIIDRNNHHNM

SEQ ID NO: 2 - protein, 20 aa, Unknown
  CHAIN: peptide used to raise anti-VCIP-RGD antibody
EGYIQNYRCRGDDSKVQEAR

SEQ ID NO: 3 - DNA, 33 bp, Artificial Sequence
  primer_bind: forward primer for VCIP
ggaggatccctcgcgccgcagccagcgccatgc

SEQ ID NO: 4 - DNA, 25 bp, Artificial Sequence
  primer_bind: reverse primer for VCIP
gtggcacctacatcatgttgtggtg

SEQ ID NO: 5 - DNA, 22 bp, Artificial Sequence
  primer_bind: forward primer for human uPAR
cttcctgaaatgcgtcaacacc

SEQ ID NO: 6 - DNA, 22 bp, Artificial Sequence
  primer_bind: reverse primer for human uPAR
tcatagctgggaaaactgaggc

SEQ ID NO: 7 - DNA, 22 bp, Artificial Sequence
  primer_bind: forward primer for _-actin
ggctgtgctatccctgtacgcc

SEQ ID NO: 8 - DNA, 22 bp, Artificial Sequence
  primer_bind: reverse primer for _-actin
gggcagtgatctccttctgcat

SEQ ID NO: 9 - DNA, 23 bp, Artificial Sequence
  primer_bind: forward primer for GAPDH
ggtctcctctgacttcaacagcg

SEQ ID NO: 10 - DNA, 24 bp, Artificial Sequence
  primer_bind: reverse primer for GAPDH
ggtactttattgatggtacatgac

SEQ ID NO: 11 - protein, 6 aa, Unknown
  CHAIN: a peptide containing RGD sequence
GRGDSP

SEQ ID NO: 12 - protein, 9 aa, Unknown
  CHAIN: HA-tag
YPYDVPDYA

SEQ ID NO: 13 - protein, 311 aa, Unknown
  CHAIN: human VCIP
MQNYKYDKAIVPESKNGGSPALNNNPRRSGSKRVLLICLDLFCLFMAGLPFLIIETSTIKPYHRGFYCNDESIKYPLKTGETINDAVLCAVGIVIAILAIITGEFYRIYYLKKSRSTIQNPYVAALYKQVGCFLFGCAISQSFTDIAKVSIGRLRPHFLSVCNPDFSQINCSEGYIQNYRCRGDDSKVQEARKSFFSGHASFSMYTMLYLVLYLQARFTWRGARLLRPLLQFTLIMMAFYTGLSRVSDHKHHPSDVLAGFAQGALVACCIVFFVSDLFKTKTTLSLPAPAIRKEILSPVDIIDRNNHHNMM

SEQ ID NO: 14 - protein, 18 aa, Unknown
  CHAIN: lipid phosphatase domain of human VCIP
DIAKVSIGRLRPHFLSVC

SEQ ID NO: 15 - protein, 18 aa, Unknown
  CHAIN: a rat peptide containing lipid phosphatase domain
DIAKYSIGRLRPHFLAVC

SEQ ID NO: 16 - protein, 18 aa, Unknown
  CHAIN: a mouse peptide containing lipid phosphatase domain
DIAKYTIGSLRPHFLAIC

SEQ ID NO: 17 - protein, 18 aa, Unknown
  CHAIN: a human peptide containing lipid phosphatase domain
DLAKYMIGRLRPNFLAVC

SEQ ID NO: 18 - protein, 18 aa, Unknown
  CHAIN: a Drosophila peptide containing lipid phosphatase domain
NIAKYSIGRLRPHFYTLC

SEQ ID NO: 19 - protein, 18 aa, C. elegans
  CHAIN: a C. elegans peptide containing lipid phosphatase domain
IVTKHVVGRLRPHFLDVC

SEQ ID NO: 20 - protein, 10 aa, Unknown
  CHAIN: a peptide containing RGD sequence
NYRCRGDDSK

SEQ ID NO: 21 - protein, 10 aa, Unknown
  CHAIN: a peptide containing a mutated RGD sequence
NYRCRADDSK

SEQ ID NO: 22 - protein, 10 aa, Unknown
  CHAIN: a peptide containing a mutated RGD sequence
NYRCRGEDSK

SEQ ID NO: 23 - protein, 13 aa, Unknown
  CHAIN: a peptide containing RGD sequence
NYRCRGDDSKVQE

SEQ ID NO: 24 - DNA, 30 bp, Artificial Sequence
  primer_bind: forward primer for phosphatase inactive or dead form of PAP2b
gccggatccatgcaaaactacaagtacgac

SEQ ID NO: 25 - DNA, 34 bp, Artificial Sequence
  primer_bind: reverse primer for phosphatase inactive or dead form of PAP2b
gaggagccaggcgccctatggacactgcggcaat

SEQ ID NO: 26 - DNA, 33 bp, Artificial Sequence
  primer_bind: forward primer for phosphatase inactive or dead form of PAP2b
tgccgcagtgtccatagggcgcctggctcctca

SEQ ID NO: 27 - DNA, 24 bp, Artificial Sequence
  primer_bind: reverse primer for phosphatase inactive or dead form of PAP2b
gcgatcgatctacatcatgttgtg

SEQ ID NO: 28 - DNA, 27 bp, Artificial Sequence
  primer_bind: forward primer for N-terminal PAP2b truncation
gccggatccatgcaaaagcgggtgctg

SEQ ID NO: 29 - DNA, 25 bp, Artificial Sequence
  primer_bind: reverse primer for N-terminal PAP2b truncation
ggtatcgataagcttctacatcatg

SEQ ID NO: 30 - DNA, 30 bp, Artificial Sequence; identical to SEQ ID NO: 24
  primer_bind: forward primer for C-terminal PAP2b truncation

SEQ ID NO: 31 - DNA, 25 bp, Artificial Sequence
  primer_bind: reverse primer for C-terminal PAP2b truncation
cgcgatcgatctacgtcgtcttagt

SEQ ID NO: 32 - protein, 6 aa, Unknown
  CHAIN: a peptide containing a RGD sequence
CRGDDS

SEQ ID NO: 33 - DNA, 24 bp, Artificial Sequence
  primer_bind: sense primer for human Alu sequence
gttgcccaagttggagtgcaatgg

SEQ ID NO: 34 - DNA, 24 bp, Artificial Sequence
  primer_bind: anti-sense primer for human Alu sequence
acaatggctcacgcctgtaatccc

SEQ ID NO: 35 - DNA, 29 bp, Artificial Sequence
  primer_bind: sense primer for mouse GAPDH
tggagtctactggtgtcttcaccaccatg

SEQ ID NO: 36 - DNA, 25 bp, Artificial Sequence
  primer_bind: anti-sense primer for mouse GAPDH
gcaggagacaacctggtcctcagtg